(19) United States
(12) Patent Application Publication
(10) Pub. No.: US 2024/0353401 A1
(43) Pub. Date: Oct. 24, 2024

(54) Compositions and methods for detecting SARS-CoV-2 spike protein

(71) Applicant: Regenacellx.SL, Barcelona (ES)

(72) Inventor: Michael EDEL, Barcelona (ES)

(73) Assignee: UNICORNFISH PTY LTD AS TRUSTEE FOR RCX UNIT TRUST, Neutral Bay (AU)

(21) Appl. No.: 18/042,138

(22) PCT Filed: Aug. 18, 2021
(86) PCT No.: PCT/IB2021/057576

§ 371 Date: Feb. 17, 2023

(30) Foreign Application Priority Data
Aug. 18, 2020 (AU) 2020902948
Jan. 22, 2021 (AU) 2021900143

(51) Int. Cl. G01N 33/543; B82Y 5/00; C12N 15/115; G01N 33/569; G01N 33/68

(52) U.S. Cl. CPC G01N 33/54388; C12N 15/115; G01N 33/56983; G01N 33/6893; B82Y 5/00; C12N 2310/16; G01N 2333/165; G01N 2800/26

(57) ABSTRACT

The present disclosure relates to compositions and methods for detecting SARS-CoV-2 spike protein and diagnosing SARS-CoV-2 infection. The present disclosure also relates to kits and devices for detecting SARS-CoV-2 spike protein and diagnosing SARS-CoV-2 infection.

### RELATED APPLICATION DATA

The present application claims priority from Australian Patent Application No. 2020902948 filed on 18 Aug. 2020 entitled “Compositions and methods for detecting SARS-COV-2 spike protein” and Australian Patent Application No. 2021900143 filed on 22 Jan. 2021 entitled “Compositions and methods for detecting SARS-COV-2 spike protein”. The entire contents of both applications are hereby incorporated by reference.

### SEQUENCE LISTING

The present application is filed together with a Sequence Listing in electronic form. The entire contents of the Sequence Listing are hereby incorporated by reference.

### FIELD

The present disclosure relates to compositions and methods for detecting SARS-CoV-2 spike protein and diagnosing SARS-CoV-2 infection. The present disclosure also relates to kits and devices for detecting SARS-CoV-2 spike protein and diagnosing SARS-CoV-2 infection.

### BACKGROUND

The severe acute respiratory syndrome coronavirus 2 (SARS-CoV-2) is the cause of coronavirus disease 2019 (COVID-19), an acute respiratory syndrome that was first identified at the end of 2019 in Wuhan, China, and quickly evolved into a pandemic. Rapid and early diagnosis of COVID-19, combined with isolation and tracking, is the main strategy of healthcare systems around the world for controlling the outbreak.

So far, the frontline response to the SARS-CoV-2 outbreak has been polymerase chain reaction (PCR) testing. PCR is the gold standard for diagnosing an infectious agent, and it has the advantage that the primers needed for such tests can be produced with relative speed as soon as the viral sequence is known. The first quantitative reverse-transcriptase-based PCR (RT-PCR) tests for detecting SARS-CoV-2 infection were designed and distributed by the World Health Organization (WHO) soon after the virus was identified. However, RT-PCR protocols are complex and expensive, and therefore are mainly suited to large, centralized diagnostic laboratories. Tests typically take 4-6 hours to complete, but the logistical requirement to ship clinical samples to a centralized laboratory means the turnaround time is 24 hours at best.

There is therefore a need for a more rapid and convenient test for detecting SARS-CoV-2.

### SUMMARY

The inventors have produced nanoparticle-based probes for detecting SARS-CoV-2 using DNA aptamers which bind to SARS-CoV-2 spike protein. The inventors found that binding of the DNA aptamers to SARS-CoV-2 spike protein induced separation of the DNA aptamers from the nanoparticles, producing a detectable signal with sensitivity in the low nanomolar range.

Thus, in one aspect, the present disclosure provides a composition comprising nanoparticles which are separably bound to a DNA aptamer that is capable of binding to SARS-CoV-2 spike protein.

In another aspect, the present disclosure provides a composition comprising nanoparticles which are separably bound to a DNA aptamer that is capable of binding to SARS-CoV-2 spike protein, wherein the DNA aptamer comprises nucleotides having a sequence which is at least 90% identical to any one of SEQ ID NOs: 1 to 8.

In another aspect, the present disclosure provides a composition comprising nanoparticles which are separably bound to a DNA aptamer that is capable of binding to SARS-CoV-2 spike protein, wherein the DNA aptamer comprises nucleotides having a sequence which is at least 70%, at least 80%, at least 90%, or at least 95% identical to any one of SEQ ID NOs: 1 to 8.

In some examples, the percent identity exists over a region that is at least about 20 nucleotides in length, or at least about 30, 40, 50, 60, 70 or more nucleotides in length. In some examples, the percent identity exists over the entire SEQ ID NO recited.

In some examples, the DNA aptamer comprises nucleotides having the sequence provided in any one of SEQ ID NOs: 1 to 8. In some examples, the DNA aptamer consists of nucleotides having the sequence provided in any one of SEQ ID NOs: 1 to 8.

In some examples, the DNA aptamer comprises nucleotides having a sequence which is at least 70%, at least 80%, at least 90%, or at least 95% identical to SEQ ID NO: 7. In some examples, the DNA aptamer comprises nucleotides having the sequence provided in SEQ ID NO: 7. In some examples, the DNA aptamer consists of nucleotides having the sequence provided in SEQ ID NO: 7.

In some examples, the DNA aptamer comprises nucleotides having a sequence which is at least 70%, at least 80%, at least 90%, or at least 95% identical to SEQ ID NO: 1. In some examples, the DNA aptamer comprises nucleotides having the sequence provided in SEQ ID NO: 1. In some examples, the DNA aptamer consists of nucleotides having the sequence provided in SEQ ID NO: 1.

In some examples, the DNA aptamer comprises nucleotides having a sequence which is at least 70%, at least 80%, at least 90%, or at least 95% identical to SEQ ID NO: 5. In some examples, the DNA aptamer comprises nucleotides having the sequence provided in SEQ ID NO: 5. In some examples, the DNA aptamer consists of nucleotides having the sequence provided in SEQ ID NO: 5.

In some examples, the DNA aptamer comprises nucleotides having a sequence which is at least 70%, at least 80%, at least 90%, or at least 95% identical to SEQ ID NO: 2. In some examples, the DNA aptamer comprises nucleotides having the sequence provided in SEQ ID NO: 2. In some examples, the DNA aptamer consists of nucleotides having the sequence provided in SEQ ID NO: 2.

In some examples, the DNA aptamer comprises nucleotides having a sequence which is at least 70%, at least 80%, at least 90%, or at least 95% identical to SEQ ID NO: 3. In some examples, the DNA aptamer comprises nucleotides having the sequence provided in SEQ ID NO: 3. In some examples, the DNA aptamer consists of nucleotides having the sequence provided in SEQ ID NO: 3.

In some examples, the DNA aptamer comprises nucleotides having a sequence which is at least 70%, at least 80%, at least 90%, or at least 95% identical to SEQ ID NO: 4. In some examples, the DNA aptamer comprises nucleotides having the sequence provided in SEQ ID NO: 4. In some examples, the DNA aptamer consists of nucleotides having the sequence provided in SEQ ID NO: 4.

In some examples, the DNA aptamer comprises nucleotides having a sequence which is at least 70%, at least 80%, at least 90%, or at least 95% identical to SEQ ID NO: 6. In some examples, the DNA aptamer comprises nucleotides having the sequence provided in SEQ ID NO: 6. In some examples, the DNA aptamer consists of nucleotides having the sequence provided in SEQ ID NO: 6.

In some examples, the DNA aptamer comprises nucleotides having a sequence which is at least 70%, at least 80%, at least 90%, or at least 95% identical to SEQ ID NO: 8. In some examples, the DNA aptamer comprises nucleotides having the sequence provided in SEQ ID NO: 8. In some examples, the DNA aptamer consists of nucleotides having the sequence provided in SEQ ID NO: 8.

In some examples, the nanoparticles are noble metal nanoparticles. Advantageously, noble metal nanoparticles such as gold and silver nanoparticles can be used to produce a colorimetric signal upon binding of the DNA aptamer to SARS-CoV-2 spike protein. In some examples, the nanoparticles comprise or consist of gold, silver, ruthenium, rhodium, palladium, osmium, iridium, platinum, mercury, rhenium, and copper. In some examples, the nanoparticles comprise or consist of silica.

In one example, the nanoparticles are gold nanoparticles. In some examples, the nanoparticles comprise or consist of gold.

The nanoparticles may be of any suitable size. In some examples, the nanoparticles have a mean diameter in the range of 5 to 100 nm. In some examples, the nanoparticles have a mean diameter in the range of 10 to 50 nm.

In some examples, the nanoparticles have a mean diameter in the range of 10 to 30 nm.

In some examples, the nanoparticles have a mean diameter of about 10 nm, about 11 nm, about 12 nm, about 13 nm, about 14 nm, about 15 nm, about 16 nm, about 17 nm, about 18 nm, about 19 nm, or about 20 nm.

In some examples, the nanoparticles have a mean diameter of about 16 nm.

In some examples, the nanoparticles have a size dispersity of less than 30%, less than 25%, less than 20%, or less than 15%. In some examples, the nanoparticles have a polydispersity index (PDI) of less than 0.3, less than 0.25, less than 0.2, or less than 0.15.

In some examples, the nanoparticles have a size dispersity of less than 20%.

In some examples, the DNA is conjugated to an affinity tag or detectable label. In some examples the DNA aptamer is biotinylated. Such aptamers are particularly useful in lateral flow assays which utilize immobilized streptavidin to capture the aptamers, for example.

In some examples, the DNA aptamer is biotinylated at its 5′ end.

In some examples, the DNA aptamer is adsorbed onto the surface of the nanoparticles. DNA aptamers can be adsorbed onto the nanoparticle surface through metal coordination interactions with DNA bases in the aptamer. This interaction between the aptamer and the nanoparticle can be disrupted by SARS-CoV-2 spike protein, which itself binds to the aptamer, thereby separating the aptamer and the nanoparticle. In some examples, separation of the aptamer and the nanoparticle produces a detectable signal. For example, the detectable signal may be a color change (e.g., of a solution) due to aggregation of the nanoparticles after separation from the DNA aptamer.

In some examples, the composition comprises
i) nanoparticles conjugated to a first polynucleotide linker; and
ii) nanoparticles conjugated to a second polynucleotide linker,
wherein the DNA aptamer comprises a region that is hybridized to the first polynucleotide linker and a region that is hybridized to the second polynucleotide linker. In these examples, the DNA aptamer aggregates the nanoparticles together by hybridizing to the two linkers that are each conjugated to separate nanoparticles. Upon binding to SARS-CoV-2, the DNA aptamer separates from the linkers (and therefore also separates from the nanoparticles) and induces disaggregation of the nanoparticles. In this case, the detectable signal may be a color change (e.g., of a solution) caused by disaggregation of the nanoparticles, for example. The color change may be the opposite color change to the above example in which the DNA aptamer is adsorbed onto the nanoparticle surface.

In some examples,
i) the first and second polynucleotide linkers are thiolated;
ii) the nanoparticles are gold nanoparticles; and
iii) the first and second polynucleotide linkers are conjugated to the gold nanoparticles via a thiol-gold bond.

In some examples, the first polynucleotide linker or the second polynucleotide linker is biotinylated.

In some examples, the DNA aptamer comprises a region that is not hybridized to either the first polynucleotide linker or the second polynucleotide linker. Advantageously, this ensures that the aptamer will preferentially bind to with SARS-CoV-2 spike protein, thus inducing the gold nanoparticles to disaggregate for signal detection. In some examples, the region that is not hybridized to either the first polynucleotide linker or the second polynucleotide linker has a length in the range of 5 to 50 nucleotides, or 10 to 30 nucleotides.

In some examples, the first polynucleotide linker and the second polynucleotide linker have a length in the range of 10 to 20 nucleotides. In some examples, the first polynucleotide linker and the second polynucleotide linker have a length in the range of 13 to 16 nucleotides.

In some examples, the region of the DNA aptamer that is hybridized to the first polynucleotide linker has a length in the range of 5 to 20 nucleotides, or 5 to 15 nucleotides, or 7 to 12 nucleotides. In some examples, the region of the DNA aptamer that is hybridized to the second polynucleotide linker has a length in the range of 5 to 20 nucleotides, or 5 to 15 nucleotides, or 7 to 12 nucleotides.

In some examples, the first polynucleotide linker comprises nucleotides having the sequence provided in SEQ ID NO: 9 and/or the second polynucleotide linker comprises nucleotides having the sequence provided in SEQ ID NO: 10.

In some examples, the first polynucleotide linker comprises nucleotides having a sequence which has no more than 5, no more than 4, no more than 3, no more than 2, or no more than 1 nucleotide variations relative to SEQ ID NO: 9.

In some examples, the second polynucleotide linker comprises nucleotides having a sequence which has no more than 5, no more than 4, no more than 3, no more than 2, or no more than 1 nucleotide variations relative to SEQ ID NO: 10.

In some examples, the composition disclosed herein is an aqueous solution.

In some examples, the nanoparticles separably bound to the DNA aptamer are present at a concentration in the range of 200 to 500 nM. In some examples, the nanoparticles separably bound to the DNA aptamer are present at a concentration in the range of 200 to 400 nM, or 250 to 350 nM. In some examples, the nanoparticles are present in the solution at a concentration of about 300 nM.

In some examples, the composition further comprises a salt. In some examples, the salt is present at a concentration which is sufficient to induce aggregation of the nanoparticles in the absence of the DNA aptamer. In some examples, the salt is present at a concentration in the range of 50 to 400 mM, 100 to 300 mM, or 150 mM to 200 mM. In some examples, the salt is present at a concentration in the range of 100 to 700 mM, or 200 to 600 mM, or 300 to 500 mM.

In some examples, the DNA aptamer is present at a concentration in the range of 200 to 500 nM. In some examples, the DNA aptamer is present at a concentration in the range of 200 to 400 nM, or 250 to 350 nM. In some examples, the nanoparticles are present in the solution at a concentration of about 300 nM.

In some examples, the concentration of DNA aptamer is previously determined or optimized for a batch of aptamer.

In some examples, the composition further comprises sodium chloride. In some examples, the composition further comprises sodium chloride at a concentration in the range of 50 to 500 mM. In some examples, the composition further comprises sodium chloride at a concentration in the range of 50 to 400 mM, 100 to 300 mM, or 150 mM to 200 mM. In some examples, the composition further comprises sodium chloride at a concentration of about 170 mM.

In some examples, the composition further comprises sodium chloride at a concentration in the range of 100 to 700 mM. In some examples, the composition further comprises sodium chloride at a concentration in the range of 200 to 600 mM, 300 to 500 mM, or 350 mM to 450 mM. In some examples, the composition further comprises sodium chloride at a concentration of about 400 mM.

In some examples, the concentration of sodium chloride is previously determined or optimized for a batch of aptamer.

In some examples, the concentration of DNA aptamer and sodium chloride are previously determined or optimized for a batch of aptamer.

In some examples, the composition is a dry composition. In some examples, the composition is dried onto a solid support. For example, the composition may be dried onto a conjugate region of a lateral assay flow device, as described herein. In some examples, the solid support comprises glass fibers, polyester, cellulose, or rayon.

The inventors have found that the compositions disclosed herein are particularly effective at detecting SARS-CoV-2 spike protein. Thus, the present disclosure also provides a method for detecting SARS-CoV-2 spike protein in a sample, comprising contacting the sample with the composition disclosed herein, wherein binding of the SARS-CoV-2 spike protein to the DNA aptamer induces separation of the DNA aptamer from the nanoparticles, thereby producing a detectable signal that is indicative of the presence of SARS-CoV-2 spike protein in the sample.

As those skilled in the art will immediately appreciate, the compositions disclosed herein can be used to diagnose SARS-CoV-2 infection in a subject, where the presence of SARS-CoV-2 spike protein in a sample from the subject is indicative of SARS-CoV-2 infection. Thus, the present disclosure also provides a method for diagnosing SARS-CoV-2 infection in a subject, comprising contacting a sample from the subject with the composition disclosed herein, wherein binding of SARS-CoV-2 spike protein to the DNA aptamer induces separation of the DNA aptamer from the nanoparticles, thereby producing a detectable signal that is indicative of SARS-CoV-2 infection.

In some examples, the detectable signal is a colorimetric signal. In some examples, the colorimetric signal is a color change. In some examples, the colorimetric signal is an increase in intensity of color. In some examples, the colorimetric signal is a decrease in intensity of color.

In some examples, the detectable signal is detected visually. In some examples, the detectable signal is detected using a spectrophotometer.

In some examples, the method is performed in solution. For example, the composition disclosed herein may be mixed with the sample in the solution in a container.

In some examples, the methods comprise measuring the solution's absorbance of light. In some examples, the methods comprise obtaining an absorbance spectrum between the wavelengths of 350 nm to 750 nm.

In some examples, the methods comprise measuring the solution's absorbance of light at a wavelength in the region of 590 nm to 650 nm and/or at a wavelength in the region of 500 nm to 540 nm. In some examples, the methods comprise measuring the solution's absorbance of light at a wavelength of 610 nm and/or at a wavelength of light in the region of 520 nm. In some examples, the methods comprise measuring the solution's absorbance of light at a wavelength of 645 nm and/or at a wavelength of light in the region of 525 nm.

In some examples, the detectable signal is an increase in the solution's absorbance of light at a wavelength in the region of 590 nm to 650 nm and/or a decrease in the solution's absorbance of light at a wavelength in the region of 500 nm to 540 nm. For example, this would occur in examples where binding of the aptamer to SARS-CoV-2 spike protein induces aggregation of the nanoparticles (e.g., when the aptamer is initially adsorbed onto the surface of the nanoparticles).

In some examples, the detectable signal is a decrease in the solution's absorbance of light at a wavelength in the region of 590 nm to 650 nm and/or an increase in the solution's absorbance of light at a wavelength in the region of 500 nm to 540 nm. For example, this would occur in examples where binding of the aptamer to SARS-CoV-2 spike protein induces disaggregation of the nanoparticles (e.g., when the aptamer is bound to the nanoparticles via polynucleotide linkers).

In some examples, the detectable signal is an increase in a ratio of the solution's absorbance of light at a wavelength of 610 compared to 520 nm. For example, if a control solution's absorbance of light at 610 nm is 0.1 AU and the control solution's absorbance of light at 520 nm is 0.4, the ratio would be 0.1/0.4, which equates to 0.25. If, in this hypothetical scenario, the solution, i.e., the solution comprising the composition disclosed herein after contacting it with the sample, has an absorbance of light at 610 nm of 0.15 AU and an absorbance of light at 520 nm of 0.3 AU, the ratio would be 0.5, which is an increase in the ratio relative to the control solution indicating that SARS-CoV-2 spike protein (and therefore virus) is present in the sample.

In some examples, the detectable signal is an increase in a ratio of the solution's absorbance of light at a wavelength of 645 compared to 525 nm.

In some examples, the methods comprise measuring the solution's absorbance of light at a wavelength in the range of 590 nm to 650 nm and a wavelength in the range of 500 nm to 540 nm, wherein an increase in a ratio of the absorbance at the wavelength in the range of 590 nm to 650 nm to the absorbance at the wavelength in the range of 500 nm to 540 nm, relative to a control solution, is indicative of the presence of SARS-CoV-2 spike protein or SARS-CoV-2 infection.

In some examples, the methods comprise measuring the solution's absorbance of light at a wavelength of 610 nm and 520 nm, wherein an increase in a ratio of absorbance at 610 nm to 520 nm, relative to a control solution, is indicative of the presence of SARS-CoV-2 spike protein or SARS-CoV-2 infection.

In some examples, the methods comprise measuring the solution's absorbance of light at a wavelength of 645 nm and 525 nm, wherein an increase in a ratio of absorbance at 645 nm to 525 nm, relative to a control solution, is indicative of the presence of SARS-CoV-2 spike protein or SARS-CoV-2 infection.

In some examples, the detectable signal is assessed using a machine learning algorithm. In some examples, the detectable signal is assessed using a machine learning algorithm to determine if SARS-CoV-2 spike protein is present in the sample. Such an algorithm uses relationships between the detectable signal observed in training data (i.e., from samples with known presence or absence of SARS-CoV-2 spike protein) to infer relationships which are then used to predict whether or not unknown samples contain SARS-CoV-2 spike protein. Thus, the machine learning algorithm may be used to predict whether or not SARS-CoV-2 spike protein is present in the sample based on the detectable signal (e.g., absorbance spectrum) produced. Thus, in some examples, an absorbance spectrum between the wavelengths of 350 nm to 750 nm is obtained and the absorbance spectrum is assessed using a machine algorithm to determine if SARS-CoV-2 spike protein is present or absent. In some examples, the machine learning algorithm is trained with a dataset comprising detectable signals from samples with a known presence or absence of SARS-CoV-2 spike protein.

In some examples, the control solution is a solution that is equivalent to the solution that has been contacted with the sample, except that the control solution has not been contacted with a sample comprising SARS-CoV-2 spike protein. In some examples, the control solution comprises nanoparticles separably bound to the DNA aptamer at a concentration that is the same or similar to the solution that has been contacted with the sample. In some examples, the control solution comprises a salt at a concentration that is the same or similar to the solution that has been contacted with the

In some examples of the methods performed in solution, the limit of detection of SARS-CoV-2 spike protein is less than 20 nM at a confidence level of 99%.

In some examples of the methods performed in solution, the sensitivity of the method is greater than 70%. Methods of determining sensitivity will be apparent to the skilled person and/or are described herein and included, for example, using a cycle threshold (Ct) value of RT-PCR. In some examples, the sensitivity of the methods performed in solution is at least 75% at a Ct value of 32. In another example, the sensitivity of the methods performed in solution is at least 75% at a Ct value of 28. For example, the sensitivity is about 77% at a Ct value of 28-29, such as 77.6% at a Ct value of 28.3. In further examples, the sensitivity of the methods performed in solution is at least 90% at a Ct value of 26.

In some examples, the methods are performed using a lateral flow assay. Suitable devices and methods for lateral flow assays are described herein.

In some examples, the sample is applied to a sample region on a lateral flow assay device. The sample can then flow through the sample region, into a conjugate region comprising the composition disclosed herein (thereby contacting the sample with the composition disclosed herein), and then onto a detection region for signal detection. In other examples, the sample is contacted with the composition disclosed herein in solution (i.e., separate to the lateral flow assay device). Subsequently, a lateral flow assay device comprising a detection region can be “dipped” directly into the resulting solution for signal detection.

In some examples of the methods performed using a lateral flow assay device, the limit of detection of SARS-CoV-2 spike protein is less than 75 nM at a confidence level of 99%.

In some examples, the method is performed using a cuvette and a spectrophotometer.

In some examples, the sample is a saliva sample. In some examples, the sample is a mouth swab. In some examples, the sample is a nasal swab. In some examples, the sample is a throat swab. Other suitable samples will be apparent to those skilled in the art.

The inventors have found that, when saliva is used as the sample, the methods disclosed herein are improved if the saliva is diluted before being contacted with the composition of the disclosure. Thus, in some examples, the saliva is diluted in an aqueous solution at a factor of at least 1 in 50, at least 1 in 100, at least 1 in 250, at least 1 in 500, at least 1 in 1000, or at least 1 in 5000 when contacted with the composition disclosed herein.

In some examples, the saliva is diluted in an aqueous solution by a factor of about 1 in 600.

In some examples, the saliva is diluted in an aqueous solution by a factor in the range of 1 in 100 to 1 in 10,000. In some examples, the saliva is diluted in an aqueous solution at a factor in the range of 1 in 600 to 1 in 10,000. In some examples, the saliva is diluted in an aqueous solution by a factor in the range of 1 in 500 to 1 in 5,000. In some examples, the saliva is diluted in an aqueous solution by a factor of about 1 in 1000. In some examples, the saliva is diluted in an aqueous solution by a factor of about 1 in 5000.

In some examples, the aqueous solution comprising the saliva sample is centrifuged prior to being contacted with the composition disclosed herein. In some examples, the aqueous solution comprising the saliva sample is filtered prior to being contacted with the composition disclosed herein.

The inventors also found that the methods disclosed herein are further improved when the subject uses a mouthwash prior to obtaining a saliva sample. Thus, in some examples, the saliva is obtained from a subject after the subject has used a mouthwash. In some examples, the volume of mouthwash used is in the range of 10 to 30 mL. Thus, in some examples, the saliva is obtained from a subject within 30 min after the subject has used a mouthwash. Thus, in some examples, the saliva is obtained from a subject within 15 min after the subject has used a mouthwash. Thus, in some examples, the saliva is obtained from a subject within 5 min after the subject has used a mouthwash.

In some examples, the mouthwash comprises an alcohol, such as ethanol. In some examples, the mouthwash comprises one or more essential oils. In some examples, the mouthwash comprises menthol, thymol, methyl salicylate, and/or eucalyptol. The mouthwash may comprise other suitable ingredients such as sorbitol, poloxamers (e.g., poloxomer 407), benzoic acid, zinc chloride, sucralose, and/or saccharin.

In some examples, the mouthwash comprises ethanol at a concentration in the range of 20% to 30%. In some examples, the mouthwash comprises eucalyptol at a concentration in the range of 0.05% to 0.15%, menthol at a concentration in the range of 0.01% to 0.1%, methyl salicylate at a concentration in the range of 0.01% to 0.1%, and thymol at a concentration in the range of 0.01% to 0.1%.

In some example, the mouthwash does not comprise an alcohol. For example, the mouthwash is alcohol-free.

In some examples, the subject has rinsed their mouth with water after using the mouthwash. In some examples, the volume of water used is in the range of 10 to 30 mL. In some example, the subject has rinsed their mouth with water one or more times after using the mouthwash. For example, the subject has rinsed their mouth with water at least 3 times after using the mouthwash.

In some examples, the subject is a human. Other non-human animals are also suitable subjects of the methods disclosed herein.

The present disclosure also provides a kit for detecting SARS-CoV-2 spike protein in a sample, the kit comprising the composition disclosed herein. Also provided is a kit for diagnosing SARS-CoV-2 infection, the kit comprising the composition disclosed herein.

In some examples, the kit comprises the composition of the disclosure in solution. For instance, in some examples, the kit comprising a container comprising the solution. In some examples, the kit comprises two or more containers comprising the solution. For example, one container of solution may be used as a control solution, and the other one or more containers may be used for contacting the composition of the disclosure with a sample.

In some examples, the container is suitable for measuring the absorbance of light of the solution in the container. In some examples, the container is a cuvette.

In some examples, the kit further comprises a spectrophotometer. Advantageously, such kits allow the presence of SARS-CoV-2 spike protein (or diagnosis of SARS-CoV-2 infection) to be detected in solution in by the subject in a point of care setting, without need for the sample to be returned to a centralized laboratory. In some examples, the spectrophotometer is a hand-held spectrophotometer and/or a portable spectrophotometer.

In some examples, the kit comprises a positive control. For example, the kit comprises a recombinant SARS-CoV-2 spike protein and/or a purified SARS-CoV-2 spike protein solution. In one example, the kit comprises a positive control comprises a container comprising a recombinant SARS-CoV-2 spike protein and/or purified SARS-CoV-2 spike protein solution.

In some examples, the kit comprises a negative control. For example, the kit comprises the composition disclosed herein which is not and/or has not been contacted with a sample.

In some examples, the kit comprises a container of mouthwash. Suitable mouthwash solutions are described herein.

In some examples, the kit comprises a pipette. In some examples the pipette is adapted to dispense a fixed volume. In some examples, the pipette is adapted to dispense a fixed volume of saliva. Such pipettes are useful for transferring and/or diluting saliva samples.

In some example, the kit comprises instructions for using the kit and/or composition disclosed herein in any method described herein.

The present disclosure also provides a lateral flow assay kit for detecting SARS-CoV-2 spike protein in a sample, the kit comprising the composition disclosed herein and a lateral flow assay device.

In some examples, the lateral flow assay kit comprises the composition of the disclosure in solution.

In some examples, the lateral flow assay kit comprises the composition of the disclosure in solution, and the lateral flow assay device comprises a detection region comprising a test zone and a control zone. Thus, in some examples of the lateral flow assay kit the composition of the disclosure is provided separately to the lateral flow assay device in the kit.

In some examples, the test zone comprises a compound that binds to the DNA aptamer. In some examples, the test zone comprises immobilized streptavidin. Such test zones are useful in examples where the aptamer or the polynucleotide linker is biotinylated.

In some examples, the control zone comprises a compound that binds to the nanoparticles. In some examples, the control zone comprises immobilized poly (diallyldimethylammonium chloride) (PDDA).

In some examples, the lateral flow assay device further comprises an absorbent region in fluid communication with, and downstream of, the detection region.

The compositions disclosed herein can also be integrated into a lateral flow assay device. Thus, the present disclosure also provides a lateral flow assay device for detecting SARS-CoV-2 spike protein in a sample, the device comprising the composition disclosed herein.

In some examples, the device comprises the composition of the disclosure dried onto a solid support.

In some examples, the device comprises the following components in fluid communication:
i) a sample region;
ii) a conjugate region downstream of the sample region; and
iii) a detection region downstream of the conjugate release region,
wherein the conjugate region comprises the composition of the disclosure and wherein the detection region comprises a test zone and a control zone.

Any example of the present disclosure herein shall be taken to apply mutatis mutandis to any other example of the disclosure unless specifically stated otherwise. For instances, examples of the compositions of the disclosure equally apply to the methods, kits and devices of the disclosure, and vice versa. Furthermore, unless the context requires otherwise, any example of the disclosure can be combined with any other example provided herein.

### KEY TO SEQUENCE LISTING

SEQ ID NO: 1—SARS-CoV-2 spike protein DNA aptamer 1
SEQ ID NO: 2—SARS-CoV-2 spike protein DNA aptamer 2
SEQ ID NO: 3—SARS-CoV-2 spike protein DNA aptamer 3
SEQ ID NO: 4—SARS-CoV-2 spike protein DNA aptamer 4
SEQ ID NO: 5—SARS-CoV-2 spike protein DNA aptamer 5
SEQ ID NO: 6—SARS-CoV-2 spike protein DNA aptamer 6
SEQ ID NO: 7—SARS-CoV-2 spike protein DNA aptamer 7
SEQ ID NO: 8—SARS-CoV-2 spike protein DNA aptamer 8
SEQ ID NO: 9—DNA linker 1
SEQ ID NO: 10—DNA linker 2

### BRIEF DESCRIPTION OF THE DRAWINGS

FIG. 1 is an electron micrograph of gold nanoparticles used to produce aptamer-nanoparticle probes for detecting SARS-CoV-2 spike protein.

FIG. 2 is a bar chart of the ratio of absorbance of light at 610 nm to 520 nm of gold nanoparticle solutions comprising various concentrations of sodium chloride and aptamer.

FIG. 3 is a bar chart of the ratio of absorbance of light at 610 nm to 520 nm of gold nanoparticle solutions comprising various concentrations of sodium chloride and aptamer. Panel A is for nanoparticles with a mean diameter of about 16 nm and panel B is for nanoparticles with a mean diameter of about 40 nm.

FIG. 4 is a line graph showing ratio of absorbance of light at 610 nm to 520 nm of DNA aptamer 1-gold nanoparticle solutions comprising various concentrations of SARS-CoV-2 spike protein.

FIG. 5 is a line graph showing ratio of absorbance of light at 610 nm to 520 nm of DNA aptamer 1-gold nanoparticle solutions comprising various concentrations of SARS-CoV-2 spike protein. The effect of centrifugation (CS) of the nanoparticles prior to adsorption with the DNA aptamer was assessed against no centrifugation (VS).

FIG. 6 is a bar chart of the ratio of absorbance of light at 610 nm to 520 nm of DNA aptamer 7-nanoparticle solutions comprising different coronavirus spike proteins.

FIG. 7 shows line graphs showing ratio of absorbance of light at 610 nm to 520 nm of DNA aptamer 1-gold nanoparticle (panel A) and DNA aptamer 7-gold nanoparticle (panel B) solutions comprising various concentrations of SARS-CoV-2 spike protein in diluted saliva samples.

FIG. 8 is a line graph showing ratio of absorbance of light at 610 nm to 520 nm of DNA aptamer 7-gold nanoparticle solutions comprising various concentrations of SARS-CoV-2 spike protein in diluted saliva samples. The sample was either centrifuged (“c”) or filtered (“F”) prior to contact with the DNA aptamer 7-gold nanoparticle solution.

FIG. 9 is a bar chart of the ratio of absorbance of light at 610 nm to 520 nm of DNA aptamer 7-nanoparticle solutions after being contacted with diluted saliva samples obtained from a subject which either did (“listerine”) or did not (“no listerine”) use a mouthwash prior to sample collection.

FIG. 10 is a photograph of a series of lateral flow assays performed on a nitrocellulose membrane dipstick. A decrease in the color intensity at the test zone is indicative of the presence of SARS-CoV-2 spike protein in the sample.

FIG. 11 is a series of line graphs showing: (A) the absorbance spectra DNA aptamer 7-nanoparticle solutions after being contacted with diluted saliva samples (B) the first derivative of the spectra in panel A, and (C) a calibration curve for determining the limit of detection.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Unless specifically defined otherwise, all technical and scientific terms used herein shall be taken to have the same meaning as commonly understood by one of ordinary skill in the art (for example, in molecular biology, immunohistochemistry, protein chemistry, and biochemistry).

Unless otherwise indicated, the techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

In general, the terms “about” and “approximately” mean within an acceptable error range for a designated value, as determined by one of ordinary skill in the art. Thus, as used herein, the term “about”, unless stated to the contrary, refers to +/−20%, or preferably +/−10%, or more preferably +/−5%, of the designated value.

As used in this disclosure, the singular forms “a,” “an,” and “the” include plural references, unless the content clearly dictates otherwise, and are used interchangeably with “at least one” and “one or more.” Thus, reference to “a DNA aptamer” can include mixtures of aptamers, and the like.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

As used herein, the term “nucleotide” refers to a deoxyribonucleotide or a ribonucleotide, or a modified form thereof, as well as an analog thereof. Nucleotides include species that include purines (e.g., adenine, hypoxanthine, guanine, and their derivatives and analogs) as well as pyrimidines (e.g., cytosine, uracil, thymine, and their derivatives and analogs). As used herein, “nucleic acid,” “oligonucleotide,” and “polynucleotide” are used interchangeably to refer to a polymer of nucleotides and include DNA, RNA, DNA/RNA hybrids and modifications of these kinds of nucleic acids, oligonucleotides and polynucleotides, wherein the attachment of various entities or moieties to the nucleotide units at any position are included. The terms “polynucleotide,” “oligonucleotide,” and “nucleic acid” include double- or single-stranded molecules as well as triple-helical molecules. Nucleic acid, oligonucleotide, and polynucleotide are broader terms than the term aptamer and, thus, the terms nucleic acid, oligonucleotide, and polynucleotide include polymers of nucleotides that are aptamers but the terms nucleic acid, oligonucleotide, and polynucleotide are not limited to aptamers. A “polynucleotide linker” is a polynucleotide that links one molecule to another through non-covalent and/or covalent bonds. For example, the DNA aptamers described herein can be, in some examples, separably bound to the gold nanoparticles via a polynucleotide linker that is conjugated to the gold nanoparticles.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions. In some examples, the protein is a fusion protein.

The term “SARS-CoV-2” refers to severe acute respiratory syndrome coronavirus 2, which is the strain of coronavirus that causes coronavirus disease 2019 (COVID-19). SARS-CoV-2 was previously referred to by its provisional name, 2019 novel coronavirus (2019-nCoV), and has also been called human coronavirus 2019 (HCoV-19 or hCoV-19). SARS-CoV-2 is a Baltimore class IV positive-sense single-stranded RNA virus that is contagious in humans. It has been described by the U.S. National Institutes of Health as a successor to SARS-CoV-1, the strain that caused the 2002-2004 SARS outbreak.

The “SARS-CoV-2 spike protein” is a transmembrane protein present on the surface of the SARS-CoV-2 virion. It is also known as the “spike glycoprotein”, the “S protein”, the “S glycoprotein”, and “E2”. The SARS-CoV-2 spike protein contains three segments: a large ectodomain, a single-pass transmembrane anchor, and a short intracellular tail. The ectodomain consists of a receptor-binding subunit S1 and a membrane-fusion subunit S2. The SARS-CoV-2 spike protein is a clove-shaped trimer with three S1 heads and a trimeric S2 stalk. During virus entry, S1 binds to a receptor on the host cell surface for viral attachment, and S2 fuses the host and viral membranes, allowing viral genomes to enter host cells. In some examples, the DNA aptamers described herein bind to the receptor-binding domain of the SARS-CoV-2 spike protein.

As used herein, the term “binding” or “binds” or “bound” refers to a physical contact between two molecules driven by chemical interactions such as electrostatic forces, hydrogen bonding and the hydrophobic effect. Two molecules may be covalently or non-covalently bound together depending on the type of chemical interaction between the molecules. Similarly, two molecules may be directly bound together or they may be indirectly bound via another molecule. In some examples, the DNA aptamer described herein is directly bound to the gold nanoparticle. In other examples, the DNA aptamer is indirectly bound to the gold nanoparticle. A molecule that is “capable of binding” another molecule is one that has affinity for the other molecule, by way of its structure, which gives it the ability to bind to the other molecule when they are brought into contact.

The term “separably bound” refers to a state in which two molecules are bound together but which can be separated by some other molecule or force acting on the two molecules. For example, the DNA aptamers described herein may be bound to gold nanoparticles by adsorption onto the nanoparticle surface through metal coordination interactions with DNA bases. This interaction between the aptamer and the nanoparticle can be disrupted by SARS-CoV-2 spike protein, which itself binds to the aptamer, thereby separating the aptamer and the nanoparticle.

“Contacting” is used in accordance with its plain ordinary meaning and refers to the process of allowing at least two distinct entities (e.g. aptamers and proteins) to become sufficiently proximal to react, interact or physically touch. It should be appreciated, however, that the resulting reaction product can be produced directly from a reaction between the added reagents or from an intermediate from one or more of the added reagents which can be produced in the reaction mixture. Contacting may include allowing two entities to react, interact, or physically touch, wherein the two entities may be the composition disclosed herein and a sample (e.g., a sample suspected of containing SARS-CoV-2 spike protein).

“Solid support” refers to any substrate having a surface to which molecules may be attached, directly or indirectly. The solid support may include any substrate material that is capable of providing physical support for the compositions described herein. The materials may be naturally occurring, synthetic, or a modification of a naturally occurring material. Suitable solid support materials may include glass fibers, polyester, cellulose, rayon, silicon, a silicon wafer chip, graphite, mirrored surfaces, laminates, membranes, ceramics, plastics (including polymers such as, e.g., poly(vinyl chloride), cyclo-olefin copolymers, agarose gels or beads, polyacrylamide, polyacrylate, polyethylene, polypropylene, poly(4-methylbutene), polystyrene, polymethacrylate, poly(ethylene terephthalate), polytetrafluoroethylene (PTFE or Teflon®), nylon, poly(vinyl butyrate)), germanium, gallium arsenide, gold, silver, Langmuir Blodgett films, a flow through chip, etc., either used by themselves or in conjunction with other materials. Additional rigid materials may be considered, such as glass, which includes silica and further includes, for example, glass that is available as Bioglass. Other materials that may be employed include porous materials, such as, for example, controlled pore glass beads, crosslinked beaded Sepharose® or agarose resins, or copolymers of crosslinked bis-acrylamide and azalactone.

“Diagnosis” or “diagnosing” in the context of the present disclosure relates to the recognition and (early) detection of a disease or clinical condition (e.g., virus infection) in a subject and may also comprise differential diagnosis. Also the assessment of the severity of a disease or clinical condition may in certain examples be encompassed by the term “diagnosis”. In accordance with the present disclosure, the presence of SARS-CoV-2 spike protein in a sample from a subject is indicative of a SARS-CoV-2 infection in that subject. Thus, the compositions and methods of the disclosure can be used to diagnose SARS-CoV-2 infection.

As used herein, the term “subject” shall be taken to mean any animal including humans, for example a mammal. Exemplary subjects include but are not limited to humans and non-human primates. In one example, the subject is a human.

### Aptamers

Aptamers, also called “nucleic acid ligands”, are nucleic acid molecules characterised by the ability to bind to a target molecule with high specificity and high affinity.

Aptamers to a given target, including those of the present disclosure, may be identified and/or produced by the method of Systematic Evolution of Ligands by Exponential enrichment (SELEX™). Aptamers and SELEX are described in Tuerk and Gold (Science, 1990, 249:505-10) and in W091/19813.

Aptamers, in general, may be DNA or RNA molecules and may be single stranded or double stranded. Aptamers for use in the compositions and methods of the present disclosure are preferably DNA aptamers. The term “DNA aptamer”, as used herein, refers to an aptamer comprising DNA or comprising modified backbone nucleic acids, such as PNA, that are derived from the DNA base sequence. In some examples, the aptamer is a single stranded DNA aptamer. The aptamer may comprise chemically modified nucleic acids, for example in which the sugar and/or phosphate and/or base is chemically modified. Such modifications may improve the stability of the aptamer or make the aptamer more resistant to degradation and may include modification at the 2′ position of ribose.

Aptamers may be synthesised by methods which are well known to the skilled person. For example, aptamers may be chemically synthesised, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer. Aptamers can be thought of as the nucleic acid equivalent of monoclonal antibodies and often have Kd's in the nM or pM range, e.g. less than one of 500 nM, 100 nM, 50 nM, 10 nM, 1 nM, 500 pM, 100 pM. As with monoclonal antibodies, they may be useful in virtually any situation in which target binding is required, including use in therapeutic and diagnostic applications, in vitro or in vivo. In vitro diagnostic applications may include use in detecting the presence or absence of a target molecule, such as SARS-CoV-2 spike protein. Aptamers for use in accordance with the present disclosure may be provided in purified or isolated form.

Aptamers according to the present disclosure may optionally have a minimum length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides.

Aptamers according to the present disclosure may optionally have a maximum length of one of 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

Aptamers according to the present disclosure may contain about 30-80 nucleotides (nts) in length. In some examples, the aptamer is about 40-80 nts, 40-65 nts, 45-55 nts, 50-80 nts, 60-80 nts, or 70-80 nts. In some examples, the nucleic acid aptamer comprising a nucleic acid motif is about 30-70 nts, 30-65 nts, 30-62 nts, 30-60 nts, 30-50 nts, or 30-40 nts. In some specific examples, the length of the aptamers may range from about 45 nts to about 55 nts.

Aptamers according to the present disclosure may optionally have a length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

Many assays can be used to qualitatively or quantitatively detect or measure binding of aptamers to SARS-CoV-2 spike protein. For example, an Enzyme-Linked Aptamer Sorbent Assay (ELASA) can be used. Assays involving amplification of the bound aptamer (e.g., qPCR) or RNA from the aptamer-bound virus (e.g., qRT-PCR) can be used. Flow cytometry methods as described in U.S. Pat. No. 5,853,984 can be used.

Microarrays, BIAcore assays, differential centrifugation, chromatography, electrophoresis, immunoprecipitation, optical biosensors, and other surface plasmon resonance assays can be used as described in WO 2011/061351. Other assays that can be used are calorimetric analysis and dot blot assays. Moreover, just as the enzyme-linked immunosorbent assay (ELISA) was adapted for aptamers in the ELASA assay, any other assays involving coronavirus spike protein-binding antibodies can be adapted for use with the DNA aptamers disclosed herein in place of the antibodies. Such assays include immunometric assays such as radioimmunoassays, flow cytometry assays, blotting applications, anisotropy, membrane assays, biosensors, and the like. Any other assays known in the art can also be used or adapted to detect or measure binding of DNA aptamers to SARS-CoV-2 spike protein. Exemplary methods for detecting binding of DNA aptamers to SARS-CoV-2 spike protein are described herein.

“Binding affinity” describes the measure of the strength of the binding or affinity of molecules to each other. Binding affinity of the aptamer herein with respect to SARS-CoV-2 is defined in terms of dissociation constant (Kd) or equilibrium dissociation constant (KD). The dissociation constant can be determined by methods known in the art and can be computed even for complex mixtures by methods such as those, for example, set forth in Caceci, M., et al., Byte (1984) 9:340-362.

Examples of measuring dissociation constants are described for example in U.S. Pat. No. 7,602,495 which describes surface Plasmon resonance analysis, U.S. Pat. Nos. 6,562,627, 6,562,627, and US 2012/00445849. In another example, the dissociation constant is established using a double-filter nitrocellulose filter binding assay such as that disclosed by Wong and Lohman, (1993). Proc. Natl. Acad. Sci. USA 90, 5428-5432.

Alternatively, or additionally a competitive binding assay for SARS-CoV-2 spike protein can be conducted with respect to substances known to bind the target or candidate. The value of the concentration at which 50% inhibition occurs (K) is, under ideal conditions, equivalent to KD. A K value can also be used to confirm that an aptamer of the present disclosure binds SARS-CoV-2 spike protein.

In some examples, the aptamers described herein bind to SARS-CoV-2 spike protein with a dissociation constant (Kd) that is lower than 100 nM, lower than 50 nM, lower than 40 nM, lower than 30 nM, lower than 20 nM, lower than 10 nM, or lower than 5 nM. The term “lower”, in this context, is in reference to a lower numerical value for the Kd, which corresponds to a higher binding affinity.

In some examples, the aptamer specifically binds to SARS-CoV-2 spike protein. For example, the aptamer may bind to SARS-CoV-2 spike protein with higher affinity than it binds to another different coronavirus spike protein.

The DNA aptamers disclosed herein can have discrete nucleic acid structures that facilitate preferential binding to SARS-CoV-2. The primary sequence of a DNA is a specific string of nucleotides (A, C, G, or T) in one dimension. The primary sequence dictates the three dimensional configuration of the aptamer.

The secondary structure of a section of DNA is represented by contact in two dimensions between specific nucleotides. Secondary structures can comprise Watson/Crick base pairs (A:T and C:G) and other base pairs of lower stability (e.g., G:T, A:C, G:A, and T:T). Secondary structures include stem loops, symmetric and asymmetric bulges, pseudoknots, and combinations of the same. In some cases, such structures can be formed in a nucleic acid sequence of no more than about 30 nucleotides. When nucleotides that are distant in the primary sequence and not thought to interact through Watson/Crick and non-Watson/Crick base pairs are in fact interacting, these interactions (which are often depicted in two dimensions) are also part of the secondary structure.

The tertiary structure of a DNA molecule is the description in space of the atoms of the DNA. Primary sequences of aptamers limit the possible tertiary structures, as do the fixed secondary structures. The DNA aptamers disclosed herein have structures in three dimensions that are comprised of a collection of DNA motifs and secondary structures, which impart the ability to bind SARS-CoV-2 on the aptamer. DNA secondary and tertiary structures include all the ways in which it is possible to describe in general terms the most stable groups of conformations that a nucleic acid compound can form.

### Nanoparticles

As used herein, the term “nanoparticles” refers to particles having a diameter between about 1 nm and 1000 nm, for example greater than about 1 nm. As would be understood by a person skilled in the art, particles are three dimensional. The nanoparticles may have any suitable shape, including, but not limited to, spherical or semi-spherical, cubic, rod like, polyhedral, rounded or semi-rounded, angular, irregular, and so forth. Where the nanoparticles do not have a uniform shape (for example, a rod, star, oval and the like) at least two of the three dimensions should be between 1 nm and 100 nm. For example, a nanotube with a diameter of 10 nm and a length of greater than 100 nm is considered a nanoparticle.

In some examples, the nanoparticles are spherical or semi-spherical.

In some examples, the nanoparticles have a mean diameter of less than about 1000 nm, less than about 900 nm, less than about 800 nm, less than about 700 nm, less than about 600 nm, less than about 500 nm, less than about 400 nm, less than about 300 nm, less than about 200 nm, less than about 100 nm, less than about 75 nm, less than about 50 nm, less than about 30 nm, or less than about 20 nm. In some examples, the nanoparticles have a mean diameter of greater than about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, or about 500 nm. The diameter may also be provided in a range between any two of these upper and/or lower values.

In some examples, the nanoparticles have a mean diameter of between about 5 to 200 nm, 10 to 100 nm, 10 to 30 nm, 10 to 20 nm, 35 nm to 1000 nm, 35 nm to 900 nm, 35 nm to 800 nm, 35 nm to 700 nm, 35 nm to 600 nm, 35 nm to 500 nm, 35 nm to 400 nm, 35 nm to 300 nm, 35 nm to 200 nm or 35 nm to 100 nm.

The diameter may be measured by any suitable means known to the skilled person, including dynamic light scattering (DLS), laser diffraction, and/or electron microscopy.

The nanoparticles may be of any suitable material known to those skilled in the art. In some examples, separation of the DNA aptamer from the nanoparticles results in a detectable signal that is indicative of the DNA aptamer binding to SARS-CoV-2 spike protein. Thus, a suitable nanoparticle material may be selected based on the desired detectable signal. The signal may be colorimetric, fluorescent, or electrochemical, for example, depending on the nanoparticle material used.

For example, noble metal nanoparticles can be used where a colorimetric signal is desired. Thus, in some examples, the nanoparticles are noble metal nanoparticles. Noble metals include gold (Au), silver (Ag), ruthenium (Ru), rhodium (Rh), palladium (Pd), osmium (Os), iridium (Ir), platinum (Pt), mercury (Hg), rhenium (Re), and copper (Cu). In some examples, the nanoparticles are gold nanoparticles or silver nanoparticles. In some examples, the nanoparticles comprise or consist of gold or silver.

Noble metal nanoparticles' interaction with light is dictated by their environment, size and physical dimensions. Oscillating electric fields of a light ray propagating near a noble metal nanoparticle interact with the free electrons causing a concerted oscillation of electron charge that is in resonance with the frequency of visible light. These resonant oscillations are known as surface plasmons. For example, for small (˜30 nm) monodisperse gold nanoparticles, the surface plasmon resonance phenomenon causes an absorption of light in the blue-green portion of the spectrum (˜450 nm) while red light (˜700 nm) is reflected, yielding a rich red color. As particle size increases, the wavelength of surface plasmon resonance related absorption shifts to longer, redder wavelengths. Red light is then absorbed, and blue light is reflected, yielding solutions with a pale blue or purple color. As particle size continues to increase toward the bulk limit, surface plasmon resonance wavelengths move into the IR portion of the spectrum and most visible wavelengths are reflected, giving the nanoparticles clear or translucent color. The surface plasmon resonance can be tuned by varying the size or shape of the nanoparticles, leading to particles with tailored optical properties for different applications.

This aggregation-color change phenomenon is also observed when excess salt is added to the nanoparticle solution. The surface charge of the nanoparticles becomes neutral, causing them to aggregate. As a result, the solution color changes from red to blue. This property can be harnessed for use of the methods disclosed herein.

Any method known in the art can be used to produce nanoparticles suitable for use in the compositions described herein. For example, there are a wide array of solution based approaches that have been developed to control the size (Sardaret al., Am. Chem. Soc. 2011, 133:8179-8190; Hussain et al., J. Am. Chem. Soc. 2005, 127:16398-16399; Jana et al., Langmuir. 2001, 17:6782-6786), shape (Grzelczak et al., Chem. Soc. Rev. 2008, 37:1783-1791) and surface functionality (Wilton-Ely JDET. Dalton Trans. 2008:25-29; Roux et al., Langmuir. 2005, 21:2526-2536; Ackerson et al., J. Am. Chem. Soc. 2005, 127:6550-6551; Daniel et al., Chem. Rev. 2004, 104:293-346).

As an example, one of the most common methods for creating gold nanoparticles is the method originally developed by Turkevich et al. (Discuss. Faraday Soc. 1951, 11:55-75) which involves treating hydrogen tetrachloroaurate (HAuCl₄) with citric acid in boiling water, where the citrate acts as both reducing and stabilizing agent. This method can be further refined by changing the gold-to-citrate ratio to control particle size (Frens Nature: Phys. Sci. 1973, 241:20-22). This protocol has been widely employed to prepare dilute solutions of moderately stable spherical gold nanoparticles with diameters of 10 to 40 nm, though larger AuNPs (e.g., 100 nm) can also be prepared.

For other methods regarding gold nanoparticles see, e.g., Schmid, G. (ed.) Clusters and Colloids (V C H, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Suitable gold nanoparticles are commercially available from, e.g., Ted Pella, Inc. (gold), Amersham Corporation (gold), Nanoprobes, Inc. (gold).

### Methods and Devices for Detecting SARS-CoV-2 Spike Protein

The compositions disclosed herein can be used for specifically, qualitatively, and/or quantitatively detecting SARS-CoV-2 virus in the context of clinical diagnosis, treatment, and/or research based on the binding of the aptamer to the SARS-CoV-2 spike protein. For example, the compositions described herein can be used for detecting SARS-CoV-2 spike protein in a test sample as an indication that the test sample contains SARS-CoV-2 virus.

As used herein, the term “sample” includes, but is not limited to, a fluid, which may comprise contains SARS-CoV-2 virus, a solution, which may comprise SARS-CoV-2 virus, and a biological sample obtained from a human or animal subject. Biological samples include but are not limited to saliva, mouth or nasal swabs, serum, blood, urine, or exhaled breath condensate. In certain examples, the sample may be fresh. It will be appreciated that a fresh sample includes, but is not limited to, a sample obtained from a subject and that is subjected to the methods described herein within several minutes, for example, less than about 5 to about 30 minutes, after the sample is obtained. In certain examples, the sample may be a stored sample. It will be appreciated that a stored sample may have been prepared and/or obtained from a subject and subjected to storage, for example in a refrigerator or freezer prior to subjecting the sample to the methods described herein. In certain examples, a sample may be used wherein the sample is not subjected to any processing (for example, dilution, filtration, concentration) prior to use in the methods described herein. In other examples, the sample is processed. Processing of the sample may involve one or more of filtration, dilution, centrifugation, distillation, extraction, concentration, fixation, inactivation of components, and the like. In some examples, the sample is diluted, filtered, or centrifuged before use.

In some examples, the sample is saliva. When saliva from a subject is used, it may be advantageous for the subject to use a mouthwash (also sometimes referred to as a “mouthrinse”) prior to obtaining the saliva sample. For example, an alcohol-containing (e.g., ethanol) mouthwash may be used. The mouthwash may comprise essential oils such as menthol, thymol, methyl salicylate, and/or eucalyptol. The mouthwash may comprise other suitable ingredients such as sorbitol, poloxamers (e.g., poloxomer 407), benzoic acid, zinc chloride, sucralose, flavorings and colorings, saccharin, and others. Suitable commercially available mouthwashes include those sold under the brand name “Listerine”. In some examples, a non-alcohol-containing mouthwash is used.

Some samples can comprise captured or pre-concentrated SARS-CoV-2 virus or protein. The term “captured or pre-concentrated” refers to SARS-CoV-2 virus or protein that has been substantially separated from an original sample. For example, a sample can be contacted with an aptamer disclosed herein or any other binding molecule, and the bound SARS-CoV-2 virus or protein can then be substantially separated from the remainder of the sample.

In some examples, the sample is an environmental sample. The term “environmental sample” refers to a sample taken or derived from any particular environment. For example, an environmental sample can be taken or derived from water (e.g., freshwater, salt water, waste water, and drinking water), soil, sewage, sludge, or an organism or tissue from an organism, such as a shellfish, that is a potential reservoir for a norovirus. An environmental sample can also be a sample taken or derived from a particular surface or object (“fomite”) in a specific location, such as a restaurant, cruise ship, hospital, nursing home, school, or other location. In certain cases, an environmental sample is taken or derived from a particular environment suspected to be a source of SARS-CoV-2.

In some examples, the sample is a food sample. The term “food sample” refers to a sample taken or derived from any food or beverage product. Examples of such food or beverage products include fresh produce, deli meats, salad bars, prepared foods (e.g., sandwiches, meat salads, casseroles), raw and cooked molluscan shellfish, and highly acidic foods such as orange juice and frozen raspberries. In certain cases, a food sample is taken from a food or beverage product suspected to be a source of SARS-CoV-2.

Some methods of detecting SARS-CoV-2 spike protein comprise contacting a sample with a composition disclosed herein, and detecting the presence of the SARS-CoV-2 spike protein in the sample. Detection of SARS-CoV-2 spike protein can indicate the presence of SARS-CoV-2 virus.

In some examples, a sample is contacted with the composition disclosed herein under conditions and for an amount of time sufficient to permit the DNA aptamer to bind to SARS-CoV-2 spike protein. For example, a sample can be incubated with at least about 10 nM, at least about 100 nM, at least about 200 nM, at least about 300 nM, or at least about 400 nM nanoparticles separably bound to the DNA aptamer. In some cases, the incubation can be done at room temperature. In other cases, it can be done at 4° C. In some cases, the incubation can be overnight. In other cases, the incubation can be for at least about 5 minutes, at least about 10 minutes, at least about 20 minutes, at least about 30 minutes, at least about 45 minutes, at least about 1 hour, at least about 2 hours, at least about 3 hours, at least about 4 hours, at least about 5 hours, at least about 6 hours, at least about 12 hours, or at least about 24 hours. In some examples, the incubation is no more than 1 hour, no more than 45 minutes, no more than 30 minutes, no more than 15 minutes, or no more than 10 minutes.

In some examples, the methods described herein can include further detection of targets other than SARS-CoV-2 spike protein as well. For example, the compositions disclosed herein may additionally comprise or can be used in conjunction with one or more other detection compounds (e.g., nucleic acid aptamers, peptide aptamers, antibodies, or the like) that preferentially bind to other molecules or agents. Other types of targets include viruses or bacteria, or antibodies that bind to SARS-CoV-2 spike protein. In some cases, the other types of viruses or bacteria detected with the one or more other detection compounds can cause symptoms in a patient that are similar to those caused by SARS-CoV-2 infection. The norovirus-binding aptamer and the other detection compound(s) can be differentially labeled so that detection of bound aptamer can indicate SARS-CoV-2 infection whereas detection of binding of the other detection compound(s) can indicate a different type of infection or condition.

Binding of the aptamers in a sample can be compared to binding of the aptamers in a control sample. The term “control sample” refers to a sample not known or suspected to include SARS-CoV-2 virus or protein. Such samples can be obtained at the same time as a test sample, clinical sample, environmental sample, or food sample suspected to include SARS-CoV-2 virus or protein, or they can be obtained on a different occasion. Such samples can be obtained from the same source or from different sources. The control sample can be the same type of sample as the test sample, clinical sample, environmental sample, or food sample to which it is being compared. For example, if the clinical sample comprises saliva, then the control sample can comprise saliva from a non-SARS-CoV-2 infected subject. In other examples, the control sample may be water, buffer, or other aqueous solution. In some examples, the detectable signal produced by the composition disclosed herein after contacting it with the sample is compared against a composition which has not been contacted with a sample, to determine if SARS-CoV-2 spike protein is present.

Multiple test samples and multiple control samples can be evaluated on multiple occasions to protect against random variation independent of the differences between the samples. A direct comparison can then be made between the test samples and the control samples to determine whether aptamer binding (i.e., the presence of SARS-CoV-2) in the test samples is increased, decreased, or the same relative to aptamer binding in the control samples. Increased binding of the aptamer in the test samples relative to the control samples indicates the presence of SARS-CoV-2 spike protein in the test samples. In some instances, increased binding is statistically significant relative to the control sample. For example, statistical significance can mean p≤0.1, p≤0.05, p≤0.01, or p≤0.001. In some cases, the term “increased binding” refers to a detectable signal resulting from aptamer binding in a test sample that is at least about 1.5-fold, at least about 2-fold, at least about 3-fold, at least about 4-fold, at least about 5-fold, at least about 10-fold, or at least about 20-fold higher than the level of signal in a control

In some examples, the methods described herein are performed in solution. For example, as described herein, certain nanoparticles exhibit a color change in the visual spectrum upon aggregation or disaggregation in solution. For example, gold nanoparticles aggregate in solution when excess salt is added to the nanoparticle solution. This is because the surface charge of the nanoparticles becomes neutral, causing them to aggregate. As a result, the solution color changes from red to blue. This property can be harnessed for use of the methods disclosed herein. For example, DNA aptamers, when adsorbed to the surface of the nanoparticle, can protect the nanoparticles from aggregating in the presence of a salt (e.g., NaCl) in solution. However, when the aptamer binds to SARS-CoV-2 spike protein, the aptamer and the nanoparticles separate, thereby leading to aggregation of the nanoparticles and a color change in the solution that can be detected (e.g., visually or by spectrophotometry).

In examples of the methods performed in solution, a sample is contacted with a solution comprising the nanoparticles separably bound to the DNA aptamer. If SARS-CoV-2 spike protein is present in the sample, a detectable signal is produced upon separation of the aptamer and the nanoparticles. The solution can be assessed for this signal and compared against a control solution. In some examples, the detectable signal is a color change. In some examples, the color change is detected visually. In some examples, the color change is detected by spectrophotometry. In some examples, the signal is detected by measuring the solution's absorbance of light at a wavelength of 610 nm and 520 nm, wherein an increase in a ratio of absorbance at 610 nm to 520 nm, relative to a control, is indicative of the presence of SARS-CoV-2 spike protein or SARS-CoV-2 infection.

Machine learning algorithms can be used to determine if SARS-CoV-2 spike protein is present in the sample. For example, detectable signals from samples such as “known samples” can be used to “train” a machine learning algorithm. A “known sample” is a sample that is pre-classified (e.g., known presence or absence of SARS-CoV-2 spike protein). The data that are derived from the detectable signals in known samples and are used to train the algorithm can be referred to as a “training data set”. Once trained, the algorithm can recognize patterns in data derived from unknown samples. The algorithm can then be used to classify an unknown sample into classes, i.e., SARS-CoV-2 spike protein present or absent. Thus, in some examples, the machine learning algorithm is a classification model, such as a binary classifier.

In some examples, the methods described herein are performed in lateral flow assay (LFA) format. LFAs, also known as “immunochromatographic strip tests”, have been a popular platform for rapid immunoassays since their introduction in the mid-1980s.

LFAs are particularly suitable where a rapid test is required or where specialized laboratory equipment is not available. In hospitals, clinics, physician offices, and clinical laboratories, LF-based tests are used for the qualitative and quantitative detection of the presence of a specific analyte in a liquid sample.

LFAs operate on the same principles as enzyme-linked immunosorbent assays (ELISA). In essence, these tests run a liquid sample along the surface of a membrane or filter paper with reactive molecules that show a visual positive or negative result depending on the presence of a particular analyte.

A lateral flow assay device, is a device configured to receive a sample at a sample region and to provide for the sample to move laterally, via, e.g. wicking, by capillary action from the sample region to a detection region. In certain examples, the lateral flow assay device further comprises one or more conjugation region(s), wherein the lateral flow assay device is configured to provide for lateral flow of a sample from a sample region to one or more conjugation region(s) prior to reaching a detection region. In related examples of a lateral flow assay device, a sample region is in contact with a conjugation region and the conjugation region is in contact with one end of a detection region such that the lateral flow assay device is configured to allow a sample to flow from the sample region, to a conjugation region and finally to a detection region. In related examples of a lateral flow assay device, a sample region is in contact with a first conjugation region, the first conjugation region is in contact with a second conjugation region, and the second conjugation region is in contact with one end of a detection region such that the lateral flow assay device is configured to allow a sample to flow from the sample region, to a first conjugation region, followed by a second conjugation region, and finally to a detection region. In certain examples of the lateral flow assay device, the device further comprises an absorbent region in contact with a detection region such that the device is configured to allow the flow of a sample from a sample region to a detection region and finally to the absorbent region.

A lateral assay device typically has a solid support onto which an optional sample region, an optional conjugate region, the detection region, and an optional absorbent region are mounted. The solid support (“backing card”) provides support for the pads and membranes of the actual assay but are otherwise not involved in the reaction or flow of the sample and analyte. Backing cards are for example made of polyvinylchloride (PVC). The assembly of pads and membranes on the backing card will typically be in a plastic housing although this is not required. The housing may have at least one opening (“sample port”) over the sample pad for application of the sample. The control and test zones are visible (e.g. via an opening or window) to detect or measure the bound label. The housing prevents the user from applying the sample anywhere except the sample pad. The housing also serves to protect the strip from inadvertent splash onto the membrane. External labelling on the housing can also be used to indicate the position of test and control lines and provide other information. Housings can be obtained as off-the-shelf cassettes or custom-designed to fit around the strip. Internal pins and bars can be used to hold the strip in place relative to the sample port and viewing window. They hold the materials in fluid communication with one another while the test strip is running.

The “sample region”, if present, receives the sample upon application and promotes the even distribution of the sample onto the detection region or conjugate region, if present. It may also influence the rate at which liquid enters the detection region, preventing flooding of the device. In addition, the sample pad may also comprise additional components such as proteins, detergents, viscosity enhancers and buffer salts in order to process the sample (e.g. separation of sample components in the case of blood samples, removal of interferences, adjustment of pH, increasing the viscosity, solubilising components and/or preventing non-specific binding between conjugate and analyte or other components or to the reaction membrane).

The “conjugate region”, if present, comprises a dried and mobilizable composition comprising the DNA aptamers and nanoparticles described. When sample flows into the conjugate region, the conjugate lifts off the conjugate region material, and moves with the sample front into the detection region. If applicable, the conjugate region will also comprise the dried and mobilizable control conjugate.

In other examples, the LFA device does not comprise a separate conjugate region. In such examples, the sample is mixed with a composition comprising the conjugate probe (i.e., the composition disclosed herein) in a separate container, prior to migration along the LFA device. Such devices may be referred to as LFA dipsticks. For example, a sample from a subject may be contacted with the composition described herein in a separate container to create a mixed solution, and then an LFA device comprising a detection region may be dipped into the solution such that it migrates along the detection region to the test and control zones.

The “detection region” is typically a membrane which comprises a test zone and control zone comprising irreversibly bound capture reagents (e.g., streptavidin). Typically, the reaction membrane is made from a polymer such as nitrocellulose, polyvinylidene fluoride, nylon or polyethersulfone. Nitrocellulose is an exemplary option for the reaction membrane. Nitrocellulose membranes bind proteins (such as antibodies or biotin-binding proteins) electrostatically through interaction of the strong dipole of the nitrate esters with strong dipoles of the peptide bonds within the protein.

The LFA device may also comprise an “absorbent region”. The absorbent region is placed at the distal end of the detection region and reserves the remaining sample. It wicks the fluid through the membrane and collects the processed liquid. Moreover, it increases the total volume of sample that can enter the detection region.

Suitable materials for a sample region, conjugation region, or a detection region that may be comprised in a lateral flow assay device described herein include, but are not limited to organic or inorganic polymers, and natural and synthetic polymers, including glass fiber, cellulose, nylon, cross-linked dextran, various chromatographic papers and nitrocellulose. It will be appreciated that suitable materials will enable a sample to flow laterally, via capillary action, along a the device described herein. In certain examples, the detection region is a nitrocellulose membrane. In certain examples, a sample region and a conjugation region may be composed of the same material. In certain examples, a lateral flow assay device comprises a sample region in capillary contact with a detection region. Suitable commercially available materials will be known to the skilled person. Commercially available materials may be used for a sample region, conjugation region, and/or detection region that may be comprised in a lateral flow assay device described herein.

The lateral flow assay device may further comprise a sample filter membrane applied to the sample region. The sample filter membrane may be composed of any suitable material including, but not limited to, a hydrophobic material capable of filtering out cells (for example blood cells) from fluids. In certain examples, a commercially available membrane, such as a Vivid Plasma Separation Membrane or a membrane similar thereto, may be used in a device described herein. Suitable sample membranes may have a filter size of about 0.22 pm to about 10 pm. In certain examples, the sample filter membrane has a filter size of less than about 10 pm, less than about 5 pm, or less than about 1 pm. In certain examples, the sample filter membrane has a size of about 0.5 pm. In certain examples, the sample filter membrane has a size of about 0.25 pm or less.

In some examples, a sample is applied to the sample region of a LFA device and the device is then incubated. Incubation comprises allowing the device to remain at a temperature, for example room temperature (eg about 20° C. to about 25° C.), such that the sample flows from the sample region to the detection region. In examples further comprising a conjugation region, incubation comprises allowing a LFD to remain at a temperature, for example room temperature (eg about 20° C. to about 25° C.), such that the sample flows from the sample region to the conjugation region followed by the detection region.

In some examples, the LFA device is incubated after applying a sample to the sample region for about 2 minutes to about 20 minutes, about 2 minutes to about 15 minutes, or about 2 minutes to about 10 minutes. In certain examples, the LFD is incubated for about 10 minutes to about 15 minutes.

In some examples, the LFA device may further comprise a control component immobilized in the control zone of the detection region. In some examples, the control component is a compound that binds to the nanoparticles in the composition described herein, such as PDDA which binds to gold nanoparticles. In related examples, the detection region of a lateral flow assay device is configured such that the sample flows past a test zone before the control zone. Related examples may further comprise inspection of the signal of a control line to confirm valid operation of a lateral flow assay device. Inspection may comprise visual confirmation of signal on a control line.

In certain examples, assessing comprises a quantitative measurement of the molecules captured on a test zone and/or control zone. In certain examples assessing may comprise semi-quantitative or qualitative assessment of a test zone, eg detection of signal above a pre-determined threshold. Suitable means of assessing a test zone will depend on the signal generated by a test zone. For example, assessing may comprise quantitatively measuring the signal from, for example, a fluorescent dye or a colloidal metal. Assessing may be carried out visually. Assessing may be carried out by a smartphone. In certain examples, assessing may comprise use of a portable fluorescence meter. Commercially available devices for measuring a signal from a lateral flow assay device will be familiar to the skilled person.

### Nucleotide Sequences

Exemplary nucleotide sequences for use in the compositions and methods described herein are provided in the table below.

TABLE 1
Exemplary nucleotide sequences

| | | SEQ |
|---|---|---|
| Name | Sequence | ID NO |
| DNA | ATCCAGAGTGACGCAGCACCGACCTTGTGCT | 1 |
| aptamer 1 | TTGGGAGTGCTGGTCCAAGGGCGTTAATGGA | |
| | CACGGTGGCTTAGT | |
| DNA | ATCCAGAGTGACGCAGCATCGAGTGGTGGGC | 2 |
| aptamer 2 | TGGTCGGGTTTGGATTCCCTTAGATGCTGGA | |
| | CACGGTGGCTTAGT | |
| DNA | ATCCAGAGTGACGCAGCACTGCGTAGGCGCG | 3 |
| aptamer 3 | GCCAATGTGTAGGATTGCTCAGGTCTGCTGG | |
| | ACACGGTGGCTTAGT | |
| DNA | ATCCAGAGTGACGCAGCATTTCATCGGGTCC | 4 |
| aptamer 4 | AAAAGGGGCTGCTCGGGATTGCGGATATGGA | |
| | CACGGTGGCTTAGT | |
| DNA | ATCCAGAGTGACGCAGCAGGACTGCTTAGGA | 5 |
| aptamer 5 | TTGCGAAGCTGAGGAGCTCCCCCGCCTTGGA | |
| | CACGGTGGCTTAGT | |
| DNA | ATCCAGAGTGACGCAGCAGTAGGGGGATTGG | 6 |
| aptamer 6 | CTCCAGGGCCTGGCTGACGGTTGCACGTGGA | |
| | CACGGTGGCTTAGT | |
| DNA | CAGCACCGACCTTGTGCTTTGGGAGTGCTGG | 7 |
| aptamer 7 | TCCAAGGGCGTTAATGGACA | |
| DNA | ATCCAGAGTGACGCAGCATTTCATCGGGTCC | 8 |
| aptamer 8 | AAAAGGGGCTGCTCGGGATTGCGGATATGGA | |
| | CACGT | |
| DNA | CCCAGGTGTCCATTA | 9 |
| linker 1 | | |
| DNA | GGTGCTGTGGGAAG | 10 |
| linker 2 | | |

As will be appreciated by a person skilled in the art, the sequences of the DNA aptamers and linkers for use in the compositions and methods of the disclosure need not be the exact sequences in Table 1. The sequences can be modified provided that the DNA aptamer is still capable of binding to SARS-CoV-2 spike protein. In some examples, the DNA aptamer comprises nucleotides having a sequence which is at least 70%, at least 75%, at least 80%, at least 85%, at least 90%, at least 95%, or at least 98% identical to any one of SEQ ID NOs: 1 to 8. In some examples, the DNA aptamer comprises nucleotides having a sequence which is 100% identical to any one of SEQ ID NOs: 1 to 8.

In other examples, the DNA aptamers described herein may contain up to 10 (e.g. including up to 9, 8, 7, 6, 5, 4, 3, 2, or 1) nucleotide variations as compared with a reference sequence, such as any one of SEQ ID NOs: 1 to 8. Positions where such variations can be introduced can be determined based on, e.g., the secondary structures of the aptamers which may be predicted using a computer algorithm, such as Mfold. For example, a base pair in a double-strand stem region may be mutated to a different base pair. Such mutations would maintain the base pair in the double-strand region at that position and thus would have no significant impact on the overall secondary structure of the aptamer. This type of mutations is well known to those skilled in the art. For example, an A-T pair may be mutated to a T-A pair. Alternatively, it may be mutated to a G-C or a C-G pair. In another example, a G-C pair may be mutated to a C-G pair. Alternatively, it may be mutated to an A-T pair or a T-A pair.

The nucleotide sequences described herein can include one or more reactive moieties. As used herein, the term reactive moiety includes any group capable of reacting with another molecule, e.g., a nucleic acid, linker as provided herein or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In examples, the internucleotide linkages in DNA are phosphodiester, phosphodiester derivatives, or a combination of both.

Alternatively or in addition, the DNA aptamers as described herein may comprise one or more locked nucleic acids (LNAs). An LNA is a modified nucleotide, in which the ribose moiety is modified with an extra bridge connecting the 2′ oxygen and 4′ carbon. This bridge “locks” the ribose in the 3′-endo (North) conformation, which is often found in the A-form duplexes. LNA nucleotides can be used in any of the DNA aptamers described herein.

### Determining Sequence Identity

Percent identity in the context of two or more nucleotide sequences, refers to the percentage of nucleotides that are the same, within a given region of the nucleotide sequences. For example, two sequences may have e.g., 60% identity, optionally 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a region in the sequences or, when specified, over the entire sequence, when compared and aligned for maximum correspondence over a comparison window, or designated region as measured using one of the following sequence comparison algorithms or by manual alignment and visual inspection. Optionally, the identity exists over a region that is at least about 20 nucleotides in length, a region that is 30, 40, 50, 60, 70 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the algorithm of Smith and Waterman (Adv. Appl. Math. 2:482c, 1970), the alignment algorithm of Needleman and Wunsch (J. Mol. Biol. 48:443, 1970), by the method of Pearson and Lipman, (Proc. Nat'l. Acad. Sci. USA 85:2444, 1988), by computerized implementations of these algorithms (e.g., GAP, BESTFIT, FASTA, and TFASTA in the Wisconsin Genetics Software Package, Genetics Computer Group), or by manual alignment and visual inspection (see, e.g., Brent et al., Current Protocols in Molecular Biology, 2003).

Two examples of algorithms that are suitable for determining percent sequence identity and sequence similarity are the BLAST and BLAST 2.0 algorithms, which are described in Altschul et al. (Nuc. Acids Res. 25:3389-3402, 1977); and Altschul et al. (J. Mol. Biol. 215:403-410, 1990), respectively. Software for performing BLAST analyses is publicly available through the National Center for Biotechnology Information.

The percent identity between two nucleotide sequences can also be determined using the algorithm of Meyers and Miller (Comput. Appl. Biosci. 4:11-17, 1988), which has been incorporated into the ALIGN program (version 2.0), using a PAM120 weight residue table, a gap length penalty of 12 and a gap penalty of 4. In addition, the percent identity between nucleotide sequences can be determined using the Needleman and Wunsch (J. Mol. Biol. 48:443, 1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

### Kits

The present disclosure also provides kits comprising the composition disclosed herein and instructions for use. Such kits can be used for, e.g., performing the detection and diagnostic methods described above. A kit can also include a label. Kits also typically contain labeling providing directions for use of the kit. Labeling generally refers to any written or recorded material that is attached to, or otherwise accompanies, a kit at any time during its manufacture, transport, sale or use. For example, the term labeling encompasses advertising leaflets and brochures, packaging materials, instructions, audio or video cassettes, computer discs, as well as writing imprinted directly on kits. Such kits may also provide a positive control, for example, a purified SARS-CoV-2 spike protein solution. The kit may also comprise a negative control, such as a solution comprising the composition disclosed herein but which is not contacted with a sample. In examples of the kits in which the composition is a dry composition, such kits may further provide a solid support on to which the composition is dried, such as a material comprising glass fibers, polyester, cellulose, or rayon.

Reagents for particular types of assays can also be provided in the kits. Thus, the kits can include a population of nanoparticles, beads (e.g., suitable for an agglutination assay or a lateral flow assay), or a plate (e.g., a plate suitable for an ELISA assay). In other examples, the kits comprise a device, such as a lateral flow assay device, an analytical rotor, or an electrochemical, optical, or opto-electronic sensor The population of nanoparticles, beads, the plate, and the devices are useful for performing an immunoassay. In examples in which the kit comprises a lateral flow assay device, the composition of the disclosure may be contained within the kit separate to the device, or it may be comprised within the device itself, for example it may be dried on to a conjugate region within the device.

In addition, the kits can include various diluents and buffers, labeled conjugates or other agents for performing the methods described above, and other signal-generating reagents, such as enzyme substrates, cofactors and chromogens. Other components of the kit can easily be determined by one of skill in the art. Such components may include coating reagents, indicator charts for colorimetric comparisons, disposable gloves, decontamination instructions, applicator sticks or containers, a sample preparatory cup, etc. In one example, a kit comprises buffers or other reagents appropriate for constituting a reaction medium in which the composition disclosed herein is contacted with the sample.

In certain examples, the kits further comprise an instruction. For example, in certain examples, the kits comprise an instruction indicating how to use the kit detect SARS-CoV-2 spike protein, or to diagnose a disease, such as a SARS-CoV-2 infection. In certain examples, the kits comprise an instruction indicating how to prepare a sample. In certain examples, the kits provide instructions for contacting the sample with the composition disclosed herein in any order prior to analyzing the sample for the presence of SARS-CoV-2 spike protein. The kits may also provide instructions for optimization of buffers, optimization of the ratios of the various components, optimization of dilution of the sample, and optimization of the order of the mixture and application steps (e.g., mix all components prior to application, mix only certain components and apply others separately).

In some examples, the kit is adapted for performing the methods of the disclosure in solution. For example, the kit may comprise a container comprising the composition disclosed herein in solution. Such kits may comprise instructions for obtaining a sample, mixing that sample with the composition of the disclosure, and then detecting a signal produced upon contacting the sample with the composition. In some examples, the container comprising the solution is suited to measuring the absorbance of light (e.g., at wavelengths in the region of 600-620 nm and/or 510 to 530 nm) by the solution using spectrophotometry. Thus, in some examples, the container comprising the composition of the disclosure is a cuvette. In some examples, the kit comprises two containers each comprising the composition of the disclosure in solution. The first container can be used for contacting with the sample, and the second container can be used as a negative control. In some examples, the kit comprises a spectrophotometer, for example a hand-held spectrophotometer, for measuring the absorbance of light of the solution. The spectrophotometer can be used where separation of the DNA aptamer and the nanoparticles in solution changes the absorbance of light at particular wavelengths of the solution. For example, where gold nanoparticles are used, separation from the DNA aptamer may cause a shift of peak absorbance from a wavelength in the blue-green spectrum (e.g., 450-550 nm) to a wavelength in the orange-red spectrum (e.g., 580-680 nm). The spectrophotometer, if present, can be suitably adapted for measuring the absorbance of light at these wavelengths.

In some examples, the kits further comprise components for obtaining, containing, preparing, measuring, and/or mixing the sample. For example, the kit may comprise a pipette for transferring a particular volume of sample, or other solution in the kit. In some examples, the pipette is adapted for dispensing saliva.

In some examples, the kits comprise a container of mouthwash. For example an alcohol-containing mouthwash for use by the subject prior to obtaining a sample from the subject's mouth (e.g., saliva).

In some examples, the kits and methods of the disclosure offer a number of advantages. For example, they can allow for simple, inexpensive, rapid, sensitive and accurate detection of SARS-CoV-2, without significant false positive or background signals. This allows for an accurate and sensitive diagnosis in a point of care setting.

### EXAMPLES

### Example 1: Preparation of Gold Nanoparticles Separably Bound to DNA Aptamer

HAuCl₄·3H₂O was obtained from Sigma (#520918-1 g, MW: 393.83 g/mol) and sodium citrate was obtained from Sigma (#S1804-500 g, MW=294, 1 g/mol). The Turkevich method was used for the preparation of gold nanoparticles of an approximate size of 15-20 nm. Specifically, a solution of 1 mM HAuCl₄·3H₂O was heated under stirring and then sodium citrate was added to a final concentration of 38.8 mM. The mixture was allowed for another 15 min and then cooled down slowly to room temperature.

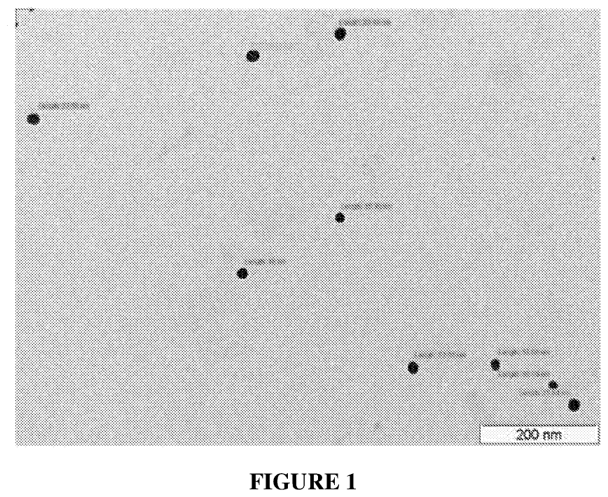

The resulting particles were analysed by electron microscopy and a representative image is shown in FIG. 1. The results of the statistical analysis of the size of the particles is shown in Table 2 below.

TABLE 2
size properties of gold nanoparticles

| | Arbitrary Line |
|---|---|
| Statistical Function | Length |
| Base Unit | nm |
| Count | 120 |
| Mean | 16.45 |
| Minimum | 7.33 |
| Maximum | 26.78 |
| Standard Deviation | 3.08 |

Table 2 shows that the gold nanoparticles had a mean diameter of 16.45 nm. FIG. 1 shows that the nanoparticles were spherical or semi-spherical in shape and had low size dispersity.

For preparing the aptamer-nanoparticle probes (i.e., the gold nanoparticles separably bound to the DNA aptamer) the gold nanoparticles were incubated with the DNA aptamer in a solution of water at room temperature for 30 mins. The optimal concentration of aptamer was determined empirically for each aptamer and batch of gold nanoparticles. Optimum concentrations of DNA aptamer were typically in the range of 200 to 500 nM.

The effect of the aptamers for preventing salt-induced aggregation of the gold nanoparticles was tested against various concentrations of sodium chloride. The optimal concentration of sodium chloride was determined empirically for each aptamer and batch of gold nanoparticles. Optimum concentrations of sodium chloride were typically in the range of 50 to 500 mM.

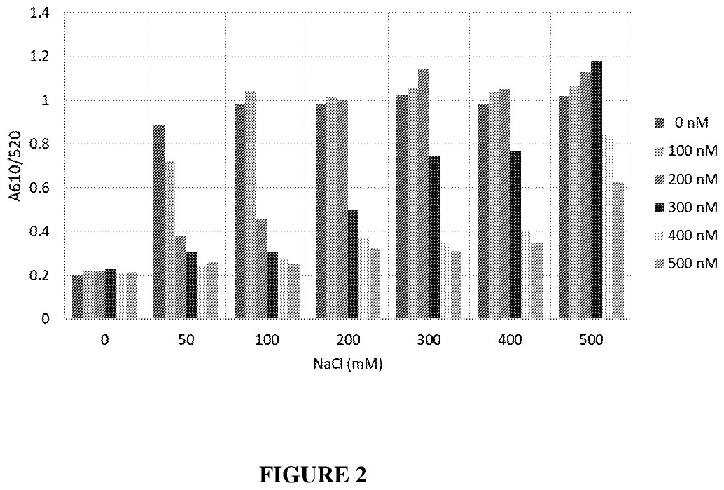

A representative experiment for determining the aptamer and sodium chloride concentration is shown in FIG. 2. In this experiment, the optimal concentration of aptamer was 300 nM and the optimal concentration of sodium chloride was 100 mM.

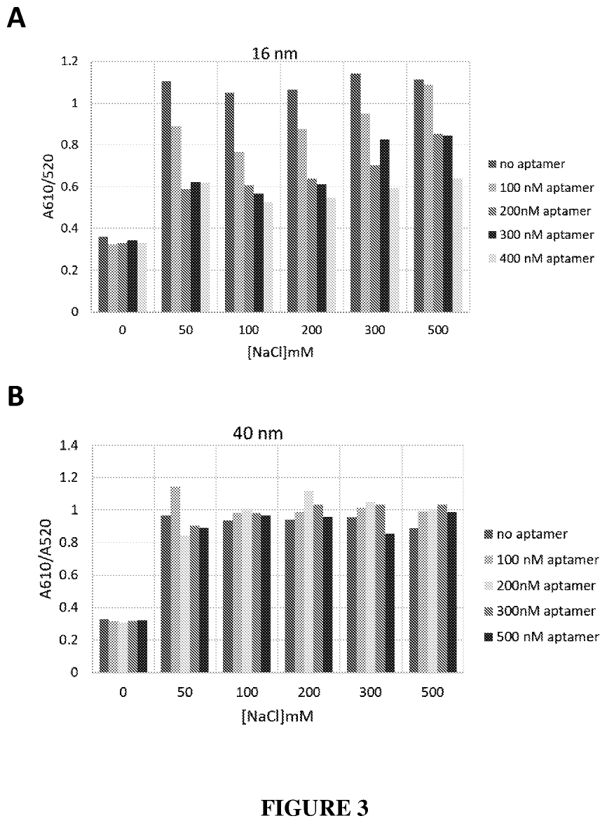

Similar experiments were also conducted to assess the effect of nanoparticle size on the prevention of salt-induced aggregation by the DNA aptamer. Specifically, nanoparticles with an approximate mean diameter of 40 nm were compared against the 16 nm nanoparticles described above. FIG. 3 shows that the 40 nm nanoparticles were not as effective for detecting the prevention of salt-induced aggregation by the DNA aptamer, relative to the 16 nm nanoparticles.

### Example 2: Detection of SARS-CoV-2 Spike Protein and Aptamer Optimisation

The aptamer-nanoparticle solutions prepared as described in Example 1 were assessed for their ability to detect binding of SARS-CoV-2 spike protein. Initially, the aptamer corresponding to SEQ ID NO:1 (DNA aptamer 1) was used.

DNA aptamer 1 was adsorbed onto the 16 nm gold nanoparticles as described in Example 1 and then was incubated for 30 min at room temperature with various concentrations of purified recombinant SARS-CoV-2 spike protein in the presence of sodium chloride. After incubation, the degree of nanoparticle aggregation was measured using the solution's ratio of absorbance of light at 610 nm to 520 nm (AU610/520).

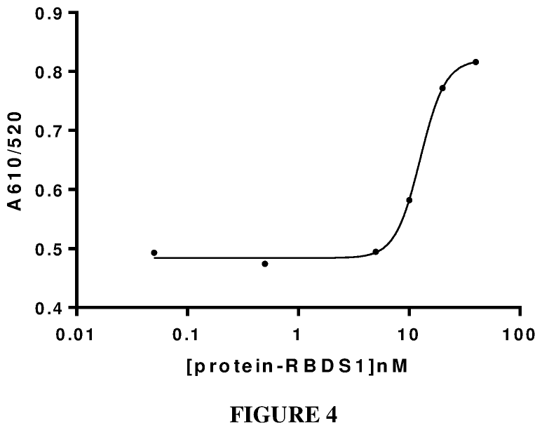

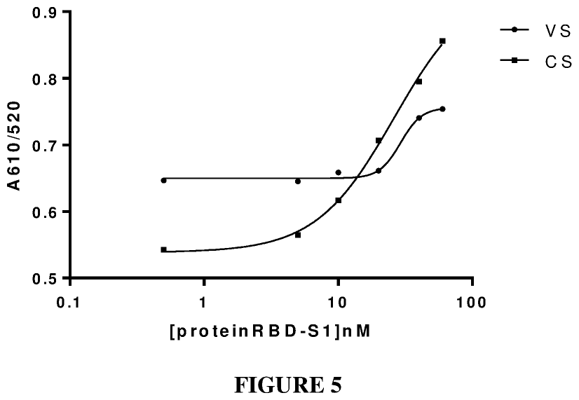

FIG. 4 shows that the AU610/520 ratio increased with increasing spike protein concentration, indicating that DNA aptamer 1 bound to the spike protein thereby resulting in salt-induced nanoparticle aggregation. The limit of detection for SARS-CoV-2 spike protein in this instance was about 5 nM. FIG. 5 shows that the limit of detection can be further enhanced when the nanoparticles are centrifuged prior to adsorption with the DNA aptamer. In this experiment, the limit of detection for SARS-CoV-2 spike protein was decreased (i.e., became more sensitive) from 8 nM in the absence of centrifugation to 2 nM when centrifuged.

The DNA aptamers 1-8 (i.e., SEQ ID NOs: 1 to 8) were then compared for their ability to bind to SARS-CoV-2 spike protein when adsorbed to the gold nanoparticles as described above. Each aptamer was adsorbed onto the 16 nm gold nanoparticles as described in Example 1 and then was incubated for 30 min at room temperature with various concentrations of purified recombinant SARS-CoV-2 spike protein (0 to 60 nM), and then sodium chloride was added to induce aggregation of the nanoparticles. The results are shown in Table 3 below.

TABLE 3
Sensitivity of nanoparticle-aptamer probes

| | | Max | Min | |
|---|---|---|---|---|
| APTAMER | LOD (nM) | AU610/520 | AU610/520 | Max − Min |
| SEQ ID NO: 1 | 8 | 0.714 | 0.348 | 0.366 |
| SEQ ID NO: 2 | 8 | 0.861 | 0.364 | 0.497 |
| SEQ ID NO: 3 | 10 | 0.777 | 0.371 | 0.406 |
| SEQ ID NO: 4 | 9 | 1.012 | 0.459 | 0.553 |
| SEQ ID NO: 5 | 9.7 | 0.708 | 0.325 | 0.386 |
| SEQ ID NO: 6 | 7.8 | 0.669 | 0.341 | 0.328 |
| SEQ ID NO: 7 | 7 | 1.112 | 0.442 | 0.672 |
| SEQ ID NO: 8 | 8.9 | 0.891 | 0.362 | 0.529 |

The sensitivity of the aptamers when adsorbed to the gold nanoparticles was assessed by subtracting the lowest AU610/520 detected (i.e., no spike protein and therefore nanoparticle aggregation inhibited) from the maximum AU610/520 (i.e., in the presence of spike protein, causing aggregation of the nanoparticles). According to this metric, DNA aptamer 7 (i.e., SEQ ID NO:7) was determined to be the most sensitive aptamer when adsorbed to the nanoparticles, with a limit of detection (LOD) for SARS-CoV-2 spike protein of about 7 nM in this instance.

The specificity of DNA aptamer 7-gold nanoparticle probe was then assessed by measuring the level of salt-induced (400 mM NaCl) nanoparticle aggregation (according to AU610/520) caused by 100 nM spike protein from the following strains of coronavirus: SARS-CoV-2, HCoV229E, HCoVNL63, MERS, and HCoVHKU.

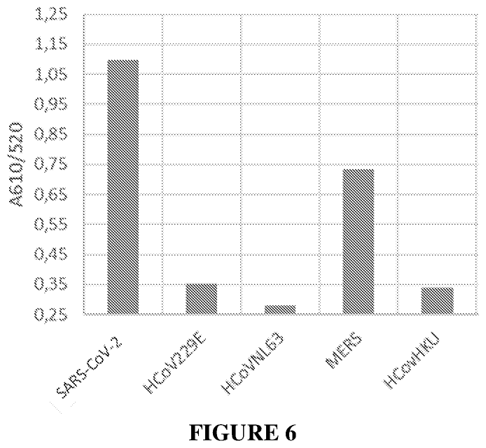

FIG. 6 shows that the DNA aptamer 7-gold nanoparticle probe was highly specific for SARS-CoV-2 spike protein, with some cross-reactivity with the MERS spike protein.

### Example 3: Detection of SARS-CoV-2 Spike Protein in Saliva

The aptamer-nanoparticle probes were assessed for their ability to detect recombinant SARS-CoV-2 spike protein in saliva. However, it was found that saliva caused the nanoparticles to aggregate, which reduced the sensitivity of the assay for detecting spike protein. Therefore, a number of different strategies were assessed for improving the assay when saliva is used as a sample: dilution; filtration; centrifugation; and use of a mouthwash.

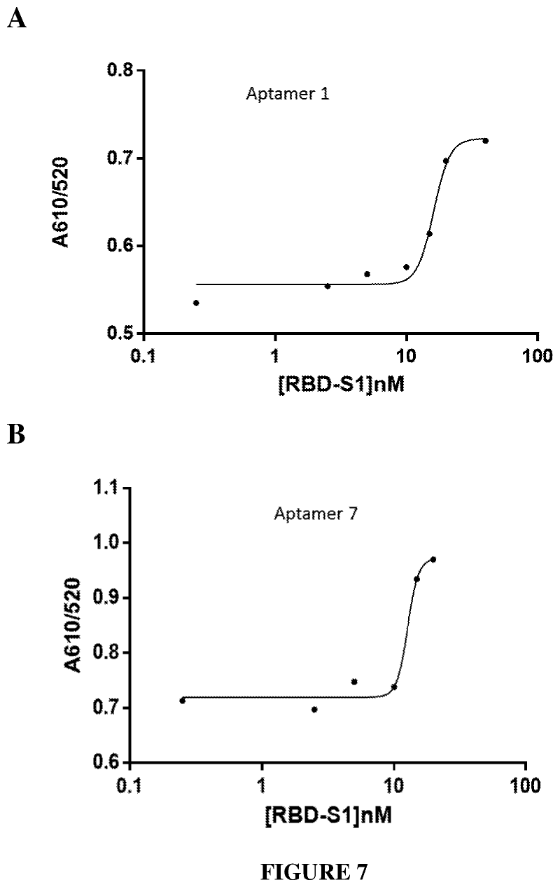

As shown in FIG. 7, dilution of the aptamer-nanoparticle probes, for both DNA aptamer 1 and DNA aptamer 7, by a factor of 1 in 600 resulted in successful detection of SARS-CoV-2 spike protein in saliva. The limit of detection was about 13 nM for DNA aptamer 1 and about 9 nM for DNA aptamer 7. Other dilution factors in the range of 1 in 100 to 1 in 10,000 were also successful.

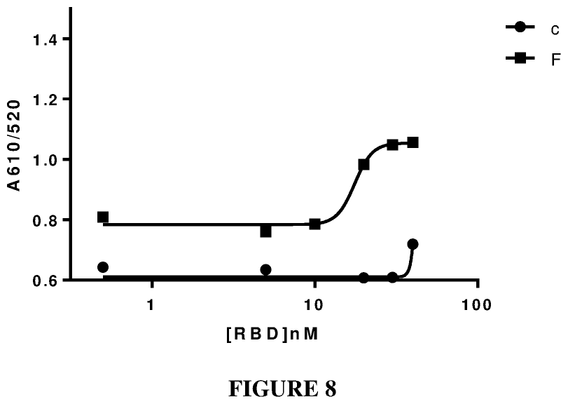

FIG. 8 shows the results of filtering and centrifuging saliva samples (diluted 1 in 300). SARS-CoV-2 spike protein was successfully detected for both of the samples. Greater detection sensitivity was observed for the samples that were filtered, prior to contacting with the DNA aptamer 7-nanoparticle solution, relative to centrifugation.

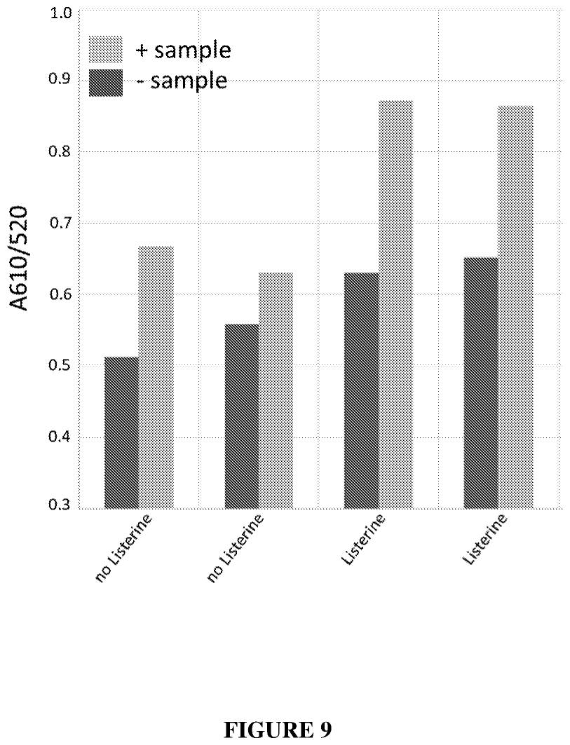

FIG. 9 shows the results of the use of a mouthwash by a subject prior to providing the saliva sample. In this case, the subject washed their mouth with 10 mL of a Listerine mouthwash, then rinsed their mouth with water, then spat into a collection vial to provide the saliva sample. Purified SARS-CoV-2 spike protein was then added to the saliva sample. The sample was diluted by a factor of 1 in 300 when contacted with DNA aptamer 7-gold nanoparticles and subsequently 400 mM NaCl was added to induce aggregation of the nanoparticles (from which the aptamer had separated upon binding to the spike protein). The use of the mouthwash improved the sensitivity of detection, as determined by the difference between the AU610/520 ratio in the presence and absence of sample.

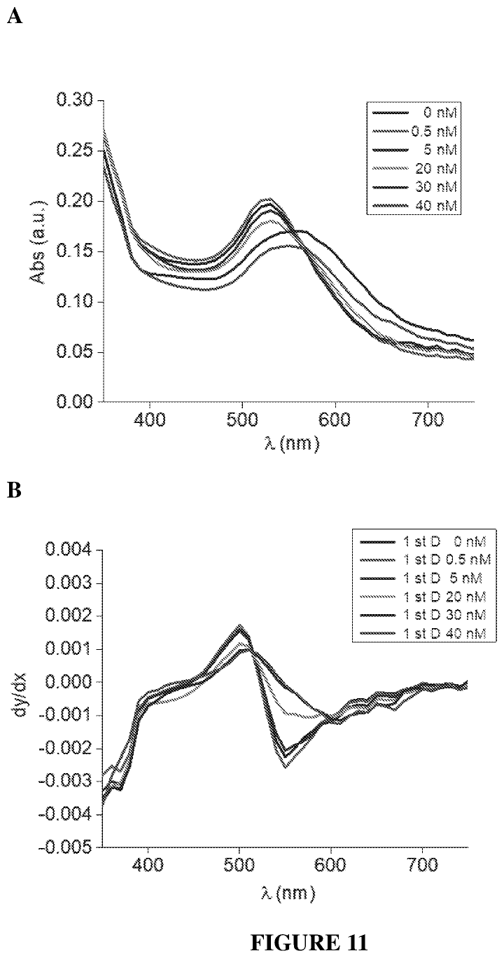

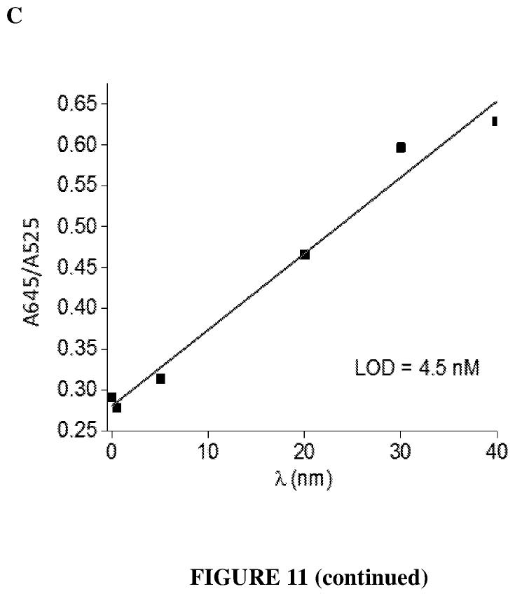

FIG. 11 shows that a limit of detection of 4.5 nM was achieved when the saliva was diluted by a factor of 1 in 1000 and the subject used a mouthwash. In this experiment, the subject performed three successive washes with a Listerine mouthwash, then rinsed their mouth with water. A saliva sample was then obtained and diluted 1 in 1000 in water before being mixed with the DNA aptamer 7-gold nanoparticles as described above and various concentration of recombinant SARS-CoV-2 spike protein (0 to 40 nM). An absorbance spectrum was recorded for each of the different concentrations of spike protein between the wavelengths of 350-750 nm (FIG. 11A). The first derivative of each spectrum was also calculated to determine the peak absorbance wavelengths (FIG. 11B). The ratio of the peak absorbance wavelengths (i.e. the absorbance at 645 nm divided by the absorbance at 525 nm) was then used to determine the limit of detection (FIG. 11C), which was calculated to be about 4.5 nM in this experiment.

### Example 4: Lateral Flow Assay for Detecting SARS-CoV-2 Spike Protein

A lateral flow assay for detecting SARS-CoV-2 spike protein was developed using the aptamer-nanoparticle probes described above. In this case, DNA aptamer 7 was biotinylated and was adsorbed onto the surface of gold nanoparticles, as described in Example 1. The resulting solution was then contacted with a sample comprising between 0 to 100 nM recombinant SARS-CoV-2 spike protein and incubated for 30 min at room temperature. A lateral flow assay dipstick device was then dipped into the mixture and the solution was allowed to flow through the dipstick. The LFA dipstick in this case comprised a nitrocellulose membrane detection region having an immobilized streptavidin test zone and a poly (diallyldimethylammonium chloride) (PDDA) control zone. The dipstick also had an absorbent region positioned downstream of the detection region to capture solution flowing through the detection region.

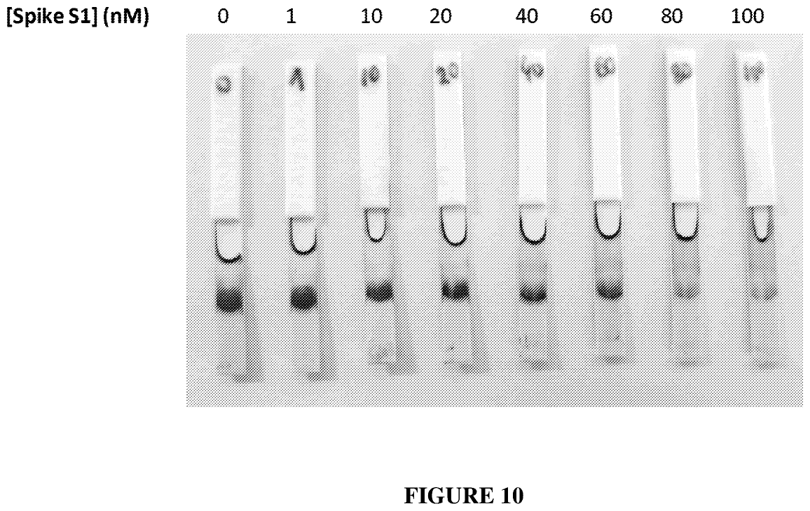

FIG. 10 shows the results of the lateral flow assays at the varying concentrations of SARS-CoV-2 spike protein. In the absence of spike protein, the gold nanoparticles remained bound to the biotinylated aptamer, resulting in a dark red spot at the streptavidin test zone (FIG. 10). Binding of the SARS-CoV-2 spike protein to the aptamer separated the aptamer from the gold nanoparticles, thereby reducing the color intensity at the control zone with increasing SARS-CoV-2 spike protein concentration. A dark line was observed at the PDDA control zone because this polymer binds to the gold nanoparticles regardless of whether the aptamer remained bound. The limit of detection for this lateral flow assay was determined to be approximately 60 nM.

### Example 5: Comparative Study with Commercially Available Rapid Test Kits

The degree of infectiousness has been shown to be related to cultivable virus. RT-PCR detection has also been shown to serve a proxy to understand infectiousness since there is a strong correlation of cycle threshold (Ct) value with the degree of cultivable virus (i.e., a higher Ct value indicates a lower viral load and a lower Ct value indicates a higher viral load). This in turn provides an understanding of the effectiveness of COVID diagnostic tests.

Using RT-PCR Ct values as a semi-quantitative measure of SARS-CoV-2 viral load, it has been shown that the level of SARS-CoV-2 RNA is greatest around symptom onset, steadily decreased during the first 10 days after illness onset and then plateaued (Table 4; data from Singanayagam et al., 2020).

TABLE 4
Geometric mean Ct values

| | GM Ct Value |
|---|---|
| After week 1 of symptom onset (days 2-7) | 28.18 |
| After week 2 (days 8-14 | 30.65 |
| After 14 days | 31.60 |

Using Ct values of RT-PCR, the sensitivity of the DNA aptamer 7-gold nanoparticle probe was compared to several commercially available rapid test kits including Panbio™ (Abbott), Standard Q (SD Biosensor), Respi-Strip (Coris Bioconcept) and NADAL® COVID-19 (Nal von Minden). Results are shown in Table 5.

TABLE 5
Comparison of sensitivity to commercially available Rapid Test Kits

| | Ct VALUE | | | | | | | |
|---|---|---|---|---|---|---|---|---|
| RAPID TEST | 14.7 | 15.3 | 23.3 | 24.2 | 26.0 | 28/29 | 31 | 32 |
| Panbio ™ | 100% | | | 83% | | 68% | | 11% |
| | | | | | | | | mean Ct |
| Standard Q | | 100% | 88% | | | | | |
| Respi-Strip | | 88% | 17% | | | | 0% | |
| NADAL ® | | 77% | 23% | | | | 0% | |
| COVID-19 | | | | | | | | |
| DNA aptamer | | | | | 91% | 77.6% | | 75.69% |
| 7-gold | | | | | | | | Ct cut- |
| nanoparticle | | | | | | | | off |

As shown in Table 5 above, although rapid antigen tests have high sensitivity at high viral loads (i.e. Ct values ≤25), at low viral loads the sensitivity drops over a wide range. In particular, the sensitivity of commercially available rapid test kits decreases from a Ct value of ˜28 (Ct 28.3) whilst the sensitivity of the DNA aptamer 7-gold nanoparticle described herein remains at 77.6%.

### Example 6: Validation Study of SARS-CoV-2 Spike Protein in Saliva

A validation study using of aptamer-nanoparticle probes to detect recombinant SARS-CoV-2 spike protein in saliva is being performed in cuvettes with a spectrophotometer.

The subject is instructed to use 15 mL of alcohol-free mouthwash (e.g., Listerine) for 30 seconds, as the first mouthwash. The subject is instructed not to gargle and asked to wash their mouth by rolling the tongue around inside of mouth. This is followed by rinsing their mouth with a mouthful of water for 30 seconds at least 3 times. All liquid and saliva is removed from the mouth.

The subject actively rolls their tongue against gums, cheeks and palate for one minute, to stimulate the production of fresh saliva. Following this a minimum of 1.5 mL of fresh saliva is collected.

The raw saliva sample is diluted. 20 μL of the raw saliva is added to a 10-mL tube pre-filled with 9980 μL of HPLC-grade water (1 in 5000 dilution) and mixed by inversion.

The following samples are measured using a cuvette:
1. A baseline measurement of the cuvette with no sample.
2. A negative control measurement using a pre-filled cuvette containing 360 μL of the aptamer-nanoparticle probes described above.
3. A positive control using a pre-filled cuvette containing 360 μL of the aptamer-nanoparticle probes described above, 40 μL of recombinant SARS-CoV-2 spike protein and 40 μL of 500 mM NaCl solution.
4. The subject's sample measurement using a pre-filled cuvette containing 360 μL of the aptamer-nanoparticle probes described above, 40 μL of the 1 in 5000 diluted saliva and 40 μL of 500 mM NaCl solution.

The UV spectrophotometer acquisition range is 450 nm to 650 nm.

All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

1. A composition comprising nanoparticles which are separably bound to a DNA aptamer that is capable of binding to SARS-CoV-2 spike protein, wherein the DNA aptamer comprises a nucleotide sequence which is at least 90% identical to any one of SEQ ID NOs: 1 to 8.

2. The composition of claim 1, wherein the DNA aptamer comprises the nucleotide sequence SEQ ID NO: 7.

3. The composition of claim 1, wherein the nanoparticles comprise one or more of the following:
i) are noble metal nanoparticles;
ii) are gold nanoparticles;
iii) have a mean diameter in the range of 10 to 30 nm; or
iv) have a size dispersity of less than 20%.

4-6. (canceled)

7. The composition of claim 1, wherein the DNA aptamer;
i) is biotinylated; or
ii) is not biotinylated or thiolated.

8. The composition of claim 7, wherein the DNA aptamer is biotinylated at its 5′ end.

9. (canceled)

10. The composition of claim 1, wherein the DNA aptamer is adsorbed onto the surface of the nanoparticles.

11. The composition of claim 1, wherein the composition comprises
i) nanoparticles conjugated to a first polynucleotide linker; and
ii) nanoparticles conjugated to a second polynucleotide linker,
wherein the DNA aptamer comprises a region that is hybridized to the first polynucleotide linker and a region that is hybridized to the second polynucleotide linker.

12. The composition of claim 11, wherein
i) the first and second polynucleotide linkers are thiolated;
ii) the nanoparticles are gold nanoparticles; and
iii) the first and second polynucleotide linkers are conjugated to the gold nanoparticles via a thiol-gold bond.

13. The composition of claim 11, wherein the composition comprises at least one of the following;
i) the first polynucleotide linker or the second polynucleotide linker is biotinylated;
ii) the DNA aptamer comprises a region that is not hybridized to either the first polynucleotide linker or the second polynucleotide linker;
iii) the first polynucleotide linker and the second polynucleotide linker have a length in the range of 10 to 20 nucleotides; or
iv) the first polynucleotide linker comprises nucleotides having the sequence provided in SEQ ID NO: 9 and/or the second polynucleotide linker comprises nucleotides having the sequence provided in SEQ ID NO: 10.

14-16. (canceled)

17. The composition of claim 1, which is;
i) an aqueous solution; or
ii) a dry composition.

18. The composition of claim 17, wherein in the aqueous solution the nanoparticles separably bound to the DNA aptamer are present at a concentration in the range of 200 to 500.

19. The composition of claim 17, wherein the aqueous solution further comprises sodium chloride at a concentration in the range of 50 to 500 nM.

20. (canceled)

21. The composition of claim 17, which is dried onto a solid support, wherein the solid support comprises glass fibers, polyester, cellulose, or rayon.

22. (canceled)

23. A method for detecting SARS-CoV-2 spike protein in a sample from a subject or for diagnosing SARS-CoV-2 infection in a subject, comprising contacting a sample from the subject with the composition of claim 1, wherein binding of the SARS-CoV-2 spike protein to the DNA aptamer induces separation of the DNA aptamer from the nanoparticles, thereby producing a detectable signal that is indicative of the presence of SARS-CoV-2 spike protein in the sample or is indicative of SARS-CoV-2 infection.

24. (canceled)

25. The method of claim 23, wherein the method comprises at leaset one of;
i) the detectable signal is a colorimetric signal;
ii) the detectable signal is assessed using a machine learning algorithm to determine if SARS-CoV-2 spike protein is present in the sample; or
iii) the method is performed in solution.

26-27. (canceled)

28. The method of claim 25, wherein, the method comprises at least one of;
i) the limit of detection of SARS-CoV-2 spike protein is less than 20 nM at a confidence level of 99%; or
ii) the method further comprises measuring the solution's absorbance of light at a wavelength of 610 nm and 520 nm, wherein an increase in a ratio of absorbance at 610 nm to 520 nm, relative to a control solution, is indicative of the presence of SARS-CoV-2 spike protein or SARS-CoV-2 infection.

29. (canceled)

30. The method of claim 23, which is performed using a lateral flow assay and wherein the limit of detection of SARS-CoV-2 spike protein is less than 75 nM at a confidence level of 99%.

31. (canceled)

32. The method of claim 23, wherein the sample is a saliva sample and wherein the method comprises at least one of:
i) the saliva is diluted in an aqueous solution at a factor of at least 1 in 50, at least 1 in 100, at least 1 in 250, at least 1 in 500, at least 1 in 1000, or at least 1 in 5000 when contacted with the composition of any one of claims 1 to 22;
ii) the saliva is diluted in an aqueous solution at a factor in the range of 1 in 600 to 1 in 10,000; or
iii) the saliva is obtained from a subject within 30 min after the subject has used a mouthwash.

33-39. (canceled)

40. A lateral flow assay kit for detecting SARS-CoV-2 spike protein in a sample, the kit comprising the composition of claim 1 and a lateral flow assay device.

41. The lateral flow assay kit of claim 40, wherein the kit comprises the composition of claim 17, and wherein the lateral flow device comprises a detection region comprising a test zone and a control zone and at least one of:
i) the test zone comprises immobilized streptavidin;
ii) the control zone comprises immobilized poly (diallyldimethylammonium chloride) (PDDA); or
iii) the device further comprises an absorbent region in fluid communication with, and downstream of, the detection region.

42-46. (canceled)


SEQUENCE LISTING (10 sequences)

SEQ ID NO: 1 - DNA, 76 bp, Artificial Sequence
  Synthetic DNA aptamer
atccagagtgacgcagcaccgaccttgtgctttgggagtgctggtccaagggcgttaatggacacggtggcttagt

SEQ ID NO: 2 - DNA, 76 bp, Artificial Sequence
  Synthetic DNA aptamer
atccagagtgacgcagcatcgagtggtgggctggtcgggtttggattcccttagatgctggacacggtggcttagt

SEQ ID NO: 3 - DNA, 77 bp, Artificial Sequence
  Synthetic DNA aptamer
atccagagtgacgcagcactgcgtaggcgcggccaatgtgtaggattgctcaggtctgctggacacggtggcttagt

SEQ ID NO: 4 - DNA, 76 bp, Artificial Sequence
  Synthetic DNA aptamer
atccagagtgacgcagcatttcatcgggtccaaaaggggctgctcgggattgcggatatggacacggtggcttagt

SEQ ID NO: 5 - DNA, 76 bp, Artificial Sequence
  Synthetic DNA aptamer
atccagagtgacgcagcaggactgcttaggattgcgaagctgaggagctcccccgccttggacacggtggcttagt

SEQ ID NO: 6 - DNA, 76 bp, Artificial Sequence
  Synthetic DNA aptamer
atccagagtgacgcagcagtagggggattggctccagggcctggctgacggttgcacgtggacacggtggcttagt

SEQ ID NO: 7 - DNA, 51 bp, Artificial Sequence
  Synthetic DNA aptamer
cagcaccgaccttgtgctttgggagtgctggtccaagggcgttaatggaca

SEQ ID NO: 8 - DNA, 67 bp, Artificial Sequence
  Synthetic DNA aptamer
atccagagtgacgcagcatttcatcgggtccaaaaggggctgctcgggattgcggatatggacacgt

SEQ ID NO: 9 - DNA, 15 bp, Artificial Sequence
  Synthetic DNA linker
cccaggtgtccatta

SEQ ID NO: 10 - DNA, 14 bp, Artificial Sequence
  Synthetic DNA linker
ggtgctgtgggaag